SEQUENCE LISTING (25 sequences)

SEQ ID NO: 1 - protein, 202 aa, Cutibacterium acnes
MTTRILLAEDIKLVAEAFEALLSVEPEFEVVARVARGDDLVTSATRLFPDVILADIDMPGMTGIEATRMLRDKGYRGRIILLTALPGSGHLHAAMAAGADGYLLKSITAPSLINAIRAVCNGQSAIDPNVAAVAMRKGPSPLNDRETEILRLVADGSSTAQIASRLYLSKGTVRNYLSTAMSKLDADSRTAAVTRAREEGWL

SEQ ID NO: 2 - protein, 389 aa, Cutibacterium acnes
MVRTRPVLDSPGRPLIASGVVLAVLLIIAVMTMVLQRDPTLTPGRRILGIVLSTVQAVGFMWLQTRMVRHGKGSRNMRATLGRVGFWATWGLVAVGVVNLWLTNSWTLFGASVATPLLLLPGVWSVVATLVTLIVGFSDLVVTHTSPLTSATVVLVTIAVSVNLYAFTKLSTALVELRSSQEEIARLRVDAERHRISRDLHDIIGRTLVATSMRQQAALHLVDRDPQRAKEQLDAAHDAITEGQHQLRSIIQAEMSTSLPDEISDATFLCDRLHIDFRMDDRGRPHKPFDSAAAAALREGITNMLKHSAAGYCHVAISPDLLTVTNDGCPQSPRPSTTPGTGIDHLRSQIEALGGSVTTSRPSRGTFRLRCSFPTRGASTTTPATEGGR

SEQ ID NO: 3 - protein, 42 aa, Cutibacterium acnes
MASTDILSPGVHRGETSHACQGVLAQHVFLDVWSLSVVNYRR

SEQ ID NO: 4 - protein, 43 aa, Cutibacterium acnes
MSVEAMQSIEMDEENSASQTVCACYSFFSWSGCCVVSQAVEAS

SEQ ID NO: 5 - protein, 573 aa, Cutibacterium acnes
MYQATWRDGTSVVVKEARHLSGLDAAGVDARVRLRHEYEALRRLDPYRAAPHPIDLIETEDGSFLIMEFLDGMTVHQEMSRFHPLIGRRPHRLSMTDFSRWCREVEDRLRNVTRVMAGCGIVHGDLHPANLIHVGHEVLVTDFESCSIDGVAVSSGIAAPWFQSDDTIPDPATTDDLHTLLVDPTCGPALVLHPNLRALISQAAVEDMMGNPTGLPHAWNLEDVTSELAKGIRASATPARADRLFPSDPYLFGHPGSEFGLMHGAAGIMAALAVTGYGVDANHVTWMKDRLATRPTLLPGLANGIEGIALGLSLCGQNDMAASLLHQSGILTITTNGSTDLTLGTGMAGRACALQSLSQRLSSKSLAHAAYTLWEQLAVAVRNTDVALPNGLFSGWEGIGLSLLASPLPDRHDLARHSLRLALATTTIVDGALFSGEGQIRWPYLGRGPAACGPLAARLDDDQTAKAVAQTCRCPLTESSGLHNGRAGLLLVLRQLVGDQDDAVRRHLMRLSWSMDRREEGTLLLGDHGLRFSSDLATGSAGALLALSRDPWRNMTRMLGICDDTCHGPLVTV

SEQ ID NO: 6 - DNA, 3775 bp, Cutibacterium acnes
  source 1..3775: mol_type = genomic DNA
ctacagccagccctcctcgcgtgcccgcgtgaccgccgctgtgcgtgagtcggcgtcgagcttcgacatcgccgtcgacaggtagttcctgacggtgcccttgctgaggtacagccgcgaggcgatctgcgcggtggaggaaccgtcggcgactaggcgcagaatctcggtttcccggtcgttgaggggcgagggtcccttgcgcatggcgaccgcggcgacgttcgggtcgatggccgactggccgttgcacaccgcccggatggcgttgatgagggagggcgcggtgatcgacttgagcaggtacccgtcggctccggcagccatcgcggcgtgcaggtgcccgcttcccggcagggcggtgagcaggatgattctgccgcggtatcccttgtcgcgaagcattcgggtggcctcgatgccggtcatcccgggcatgtcgatgtcggccaggatcacgtcggggaacaggcgggtggcggatgtgaccaggtcgtcgccgcgtgccacccgtgcgacgacctcgaactccggttccaccgagagcagtgcctcgaaggcctctgccaccagtttgatgtcctcggcgaggaggattctcgtcgtcatcgtcctccttcggtcgcgggtgtggtcgtggacgccccccgggtcgggaaggagcagcggagccggaaggtgccccgcgagggccgcgacgtcgtcaccgacccaccgagcgcctcgatctgggaccggagatggtcgattccggtgcccggtgtggtggaggggcgaggtgattgcggacaaccgtcgttggtgacggtcaggaggtccggggagatggcgacgtggcagtagcccgccgcgctgtgcttgagcatgttcgtgattccctcgcgcagggccgccgccgccgccgagtcgaagggtttgtgcggtctgccgcggtcatccatgcggaagtcgatgtggagtcggtcgcacaggaatgtggcgtcgctgatctcgtcggggaggctggtgctcatctcggcctggatgatggaacgcaactggtgctgcccctcggtgattgcgtcatgggcggcgtcgagctgttccttggcccgttgcgggtcgcggtcgacgaggtggagggccgcctgctggcgcatcgacgtggcgaccagggtgcgaccgatgatgtcgtggaggtcccgcgatatccggtgacgctcggcgtccacgcgcagccgggcgatctcctcctgggaggagcggagttcaacgagtgccgtcgacagcttcgtgaatgcgtagaggttcaccgacaccgcgatggtcaccagcacgaccgtggctgaggtcagtggcgaggtgtgggtgaccacgaggtcggagaaaccgacgatcagggtcacgagggtggccaccacactccacacccctggcaggagaaggaggggcgtggcgacggaggccccgaagagggtccacgagttggtcagccacagattgacgactcccactgcgacgagtccccacgtggcccagaaaccgacccttccaagggtcgcacgcatgttgcgcgaacccttgccgtgacgaaccatgcgggtctgcagccacatgaatccgacggcctggaccgtggacaggacgataccgagaatgcggcgtccgggggtgagagtggggtcgcgctgaagcaccatcgtcatcaccgcgatgatgagcaggaccgccaggacgactccggaggcgatgagcggtcttccgggactgtccaggacggggcgtgtgcgtaccacgggggacagggtagtcgacggcgtctcacacgatcaagatgcttccaccgcttgggagactacgcagcatcctgaccacgagaagaagctgtaacaagcgcaaactgtctggctggcgctgttttcctcgtccatctcgatggattgcatggcctcaacggacattctgtctcctggtgttcatcgtggtgaaacgagccacgcctgccaaggcgttttggctcagcatgttttcctcgatgtttggagtctttcagttgtgaactatcggcgctagatcaaactgtcactagtgggccatgacaggtgtcatcgcatattccgagcattcgtgtcatgttccgccatgggtccctgctcagggcgaggagggcgccggctgaccccgtcgcgagatcggatgagaaacgcaggccatgatcacccagcagcaatgtgccttcctcgcgacggtccatcgaccacgacagtctcatgaggtggcggcgaacggcgtcgtcttggtctcccacgagctggcggaggacgagtaggagacctgcgcggccattgtggagaccggatgactcagtcaggggacaacgacaggtctgtgccacggccttggccgtctggtcatcatcaagcctggcggcgagcgggccacatgccgcgggaccacgccccagatatggccatcggatctgtccctcgcctgagaagagcgcaccgtcaacaatcgttgtcgtggccagggccagtcggagtgagtggcgggcgagatcatgacgatccggcaggggagaggcaagcagggagagcccgatcccctcccaaccggagaacaggccgttcggcaaggccacatcagtgttccgcacagccacggcgagttgttcccagagggtatatgcagcgtgggcgagagatttcgatgacagtcgttgtgagagggactgcagcgcgcacgctcggcccgccataccagtaccgagggtgaggtcagtcgatccgttcgttgtgatcgtgagaattccggactggtggagcagggatgcagccatgtcgttctgaccgcacagggagagacccagggcaattccctcgattccgttggccaaccccggcagcagggtcgggcgggtggccaagcggtccttcatccaggtcacgtgattcgcgtcaacaccgtacccagtcacggcaagagcagccatgattccggcggcgccatgcatcaggccgaattctgaacctggatgcccgaacaggtaggggtctgagggaaagaggcggtcggctcgtgcgggagtggctgatgcccggatccccttggcgagttcgcttgtcacgtcctcgaggttccaggcatgtggtaaaccagtcggatttcccatcatgtcctcgactgcggcctggctgatgagcgcgcgaagatttggatggaggacaagcgcggggccgcaagtcggatccacgagaagagtgtggagatcgtcagtcgtcgccggatcgggaatcgtgtcgtccgactggaaccagggagcggcgatccctgacgagacggcgaccccgtcgattgagcaggattcaaagtcagtaaccaaaacctcatggccgacatgtatcagattcgcgggatgaagatccccgtgaacgattccgcacccggccatgaccctcgtgacattcctcaagcgatcttcgacctctcggcaccagcgtgaaaaatcggtcatcgatagccgatgaggtcgcctaccaatcagaggatggaacctcgacatctcctggtggactgtcattccgtcaaggaattccatgatcaggaacgagccgtcctcggtctcaatgagatcaatgggatggggagcggcccgataggggtccagtcttctcagggcctcgtactcgtgacgcagccgcactcgggcgtcgacacccgccgcatcaagtccggacaggtgccgggcctccttgacgactaccgatgtgccgtctctccatgttgcctgatacac

SEQ ID NO: 7 - protein, 202 aa, Cutibacterium acnes; identical to SEQ ID NO: 1

SEQ ID NO: 8 - protein, 389 aa, Cutibacterium acnes; identical to SEQ ID NO: 2

SEQ ID NO: 9 - protein, 42 aa, Cutibacterium acnes; identical to SEQ ID NO: 3

SEQ ID NO: 10 - protein, 43 aa, Cutibacterium acnes; identical to SEQ ID NO: 4

SEQ ID NO: 11 - protein, 573 aa, Cutibacterium acnes; identical to SEQ ID NO: 5

SEQ ID NO: 12 - protein, 573 aa, Cutibacterium acnes
MYQATWRDGTSVVVKEARHLSGLDAAGVDARVRLRHEYEALRRLDPYRAAPHPIDLIETEDGSFLIMEFLDGMTVHQEMSRFHPLISRRPHRLSMTDFSRWCREVEDRLRNVTRVMAGCGIVHGDLHPANLIHVGHEVLVTDFESCSIDGVAVSSGIAAPWFQSDDTIPDPATTDDLHTLLVDPTCGPALVPHPNLRALISQAAVEDMMGNPTGLPHAWNLEDMTSELAKGIRASATPARADRLFPSDPYLFGHPGSEFGLMHGAAGIMAALAVTGYGVDANHVTWMKDRLATRPTLLPGLANGIEGIALGLSLCGQNDMAASLLHQSGILTITTNGSTDLTLGTGMAGRACALQSLSQRLSSKSLAHAAYTLWEQLAVAVRNTDVALPNGLFSGWEGIGLSLLASPLPDRHDLARHSLRLALATTTIVDGALFSGEGQIRWPYLGRGPAACGPLAARLDDDQTAKAVAQTCRCPLTESSGLHNGRAGLLLVLRQLVGDQDDAVRRHLMRLSWSMDRREEGTLLLGDHGLRFSSDLATGSAGALLALSRDPWRNMTRMLGICDDTCHGPLVTV

SEQ ID NO: 13 - protein, 573 aa, Cutibacterium acnes
MYQATWRDGTSVVVKEARHLSGLDAAGVDARVRLRHEYEALRRLDPYRAAPHPIDLIETEDGSFLIMEFLDGMTVHQEMSRFHPLISRRPHRLSMTDFSRWCREVEDRLRNVTRVMAGCGIVHGDLHPANLIHVGHEVLVTDFESCSIDGVAVSSGIAAPWFQSDDTIPDPATTDDLHTLLVDPTCGPALVPHPNLRALISQAAVEDMMGNPTGLPHAWNLEDMTSELAKGIRASATPARADRLFPSDPYLFGHPGSEFGLMHGAAGIMAALAVTGYGVDANHVTWMKDRLATRPTLLPGLANGIEGIALGLSLCGQNDMAASLLHQSGILTITTNGSTDLTLGTGMAGRACALQSLSQRLSSKSLAHAAYTLWEQLAVAMRNTDVALPNGLFSGWEGIGLSLLASPLPDRHDLARHSLRLALATTTIVDGALFSGEGQIRWPYLGRGPAACGPLAARLDDDQTAKAVAQTCRCPLTESSGLHNGRAGLLLVLRQLVGDQDDAVRRHLMRLSWSMDRREEGTLLLGDHGLRFSSDLATGSAGALLALSRDPWRNMTRMLGICDDTCHGPLVTV

SEQ ID NO: 14 - protein, 573 aa, Cutibacterium acnes
MYQATWRDGTSVVVKEARHLSGLDAAGVDARVRLRHEYEALRRLDPYRAAPHPIDLIETEDGSFLIMEFLDGMTVHQEMSRFHPLISRRPHRLSMTDFSRWCREVEDRLRNVTRVMAGCGIVHGDLHPANLIHVGHEVLVTDFESCSIDGVAVSSGIAAPWFQSDDTIPDPATTDDLHTLLVDPTCGPALVPHPNLRALISQAAVEDMMGNPTSLPHAWNLEDMTSELAKGIRASATPARADRLFPSDPYLFGHPGSEFGLMHGAAGIMAALAVTGYGVDANHVTWMKDRLATRPTLLPGLANGIEGIALGLSLCGQNDMAASLLHQSGILTITTNGSTDLTLGTGMAGRACALQSLSQRLSSKSLAHAAYTLWEQLAVAVRNTDVALPNGLFSGWEGIGLSLLASPLPDRHDLARHSLRLALATTTIVDGALFSGEGQIRWPYLGRGPAACGPLAARLDDDQTAKAVAQTCRCPLTESSGLHNGRAGLLLVLRQLVGDQDDAVRRHLMRLSWSMDRREEGTLLLGDHGLRFSSDLATGSAGALLALSRDPWRNMTRMLGICDDTCHGPLVTV

SEQ ID NO: 15 - DNA, 3775 bp, Cutibacterium acnes; identical to SEQ ID NO: 6
  source 1..3775: mol_type = genomic DNA

SEQ ID NO: 16 - DNA, 23 bp, synthetic construct
  source 1..23: mol_type = other DNA
ggttccatcctctgattggtagg

SEQ ID NO: 17 - DNA, 21 bp, synthetic construct
  source 1..21: mol_type = other DNA
ccctcgtgacattcctcaagc

SEQ ID NO: 18 - DNA, 21 bp, synthetic construct
  source 1..21: mol_type = other DNA
ccagttgcgttccatcatcca

SEQ ID NO: 19 - DNA, 20 bp, synthetic construct
  source 1..20: mol_type = other DNA
ggaagtcgatgtggagtcgg

SEQ ID NO: 20 - DNA, 20 bp, synthetic construct
  source 1..20: mol_type = other DNA
gcaacccgacatcctcatta

SEQ ID NO: 21 - DNA, 20 bp, synthetic construct
  source 1..20: mol_type = other DNA
agtcgaagaagtcgctcagg

SEQ ID NO: 22 - DNA, 21 bp, synthetic construct
  source 1..21: mol_type = other DNA
gaagaatcccgctccatttcc

SEQ ID NO: 23 - DNA, 20 bp, synthetic construct
  source 1..20: mol_type = other DNA
cctttcttgtagccgagcag

SEQ ID NO: 24 - DNA, 22 bp, synthetic construct
  source 1..22: mol_type = other DNA
ggcaagacaaacgaggtaggag

SEQ ID NO: 25 - DNA, 23 bp, synthetic construct
  source 1..23: mol_type = other DNA
gatggattgtggttggagtctcc

(19) United States
(12) Patent Application Publication
(10) Pub. No.: US 2023/0310515 A1
(43) Pub. Date: Oct. 5, 2023

(54) COMPOSITIONS AND METHODS FOR TREATING SKIN INFECTIONS AND OTHER DISEASES

(71) Applicant: The Regents of the University of California, Oakland, CA (US)

(72) Inventors: Huiying Li, Los Angeles, CA (US); Mizue Naito, Los Angeles, CA (US)

(21) Appl. No.: 17/993,329

(22) Filed: Nov. 23, 2022

Related U.S. Application Data
(62) Division of application No. 16/935,899, filed on Jul. 22, 2020, now abandoned.
Application No. 16/935,899 is a continuation-in-part of application No. PCT/US20/42542, filed on Jul. 17, 2020.
(60) Provisional application No. 62/875,641, filed on Jul. 18, 2019.
(60) Provisional application No. 62/877,045, filed on Jul. 22, 2019.

(51) Int. Cl. A61K 35/74; A61P 17/10; A61P 31/04; A61K 9/00

(52) U.S. Cl. CPC A61K 35/74; A61K 9/0014; A61P 31/04; A61P 17/10

(57) ABSTRACT

Provided herein are methods and compositions for promoting skin health.

### RELATED APPLICATIONS

This application is a divisional of U.S. application Ser. No. 16/935899, filed Jul. 22, 2020, which claims priority to U.S. Provisional Application No. 62/877045, filed Jul. 22, 2019, and is a continuation-in-part to Application PCT/US20/42542, filed Jul. 17, 2020, which claims the benefit of U.S. Provisional Application No. 62/875641, filed Jul. 18, 2019, the entire contents of each of which are incorporated by reference herein in their entirety.

### GOVERNMENT SUPPORT

This invention was made with government support under Grant Number GM099530, awarded by the National Institutes of Health. The government has certain rights in the invention.

### BACKGROUND

The skin is the largest organ in the human body and functions as the first line of defense by providing a protective barrier between the environment and inner body. The skin harbors several hundreds of resident microorganisms, which function in communities and protect the body from invasion of pathogens. Several studies have shown that shifts in the skin microbiota are associated with various skin diseases.

### SUMMARY

Provided herein are methods and compositions useful in the treatment of infections caused by Gram-positive bacteria. In some aspects, provided herein are methods of treating or preventing a disease or condition (e.g., a skin disease or a disease associated with bacteria disclosed herein) in a subject in need thereof by administering a composition comprising agent X, a composition comprising a bacteria (or strain thereof) comprising Locus 4 (e.g., a P. acnes or P. avidum strain comprising Locus 4), a composition comprising a bacteria (or strain thereof) that expresses a peptide of any one of SEQ ID NOs: 1-5, 7-14, or any other amino acid disclosed herein, or a composition comprising a bacteria (or strain thereof) that comprises the nucleic acid sequence 6, 15 or any other nucleic acid sequence disclosed herein. In some embodiments, the methods comprise administering to the subject a composition comprising a Propionibacterium bacterium that expresses agent X to the subject. Also provided herein are methods of maintaining healthy skin in a subject in need thereof by administering a composition or agent (e.g., agent X) disclosed herein, a composition comprising a strain of bacteria comprising Locus 4 (e.g., a P. acnes or P. avidum strain comprising Locus 4), or a composition comprising a strain of bacteria that expresses a peptides of any one of SEQ ID NOs: 1-5 or 7-14 to the subject. The compositions disclosed herein may comprise live, replication competent bacteria. The compositions disclosed herein may be heat treated, tyndallized and/or supernatant-derived.

In some aspects, provided herein are methods of maintaining healthy skin in a subject in need thereof by administering a composition comprising agent X to the subject or administering a composition comprising a Propionibacterium bacterium that expresses agent X to the subject. Agent X may be encoded by Locus 4. Agent X may comprise at least one (e.g., at least two, at least three, at least four, at least five, at least six, or at least seven) peptide with an amino acid sequence with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with the nucleic acid sequence of Locus 4. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with any one of SEQ ID NOs: 1-5 or 7-14.

The strain of bacteria may be any strain which comprises Locus 4. The strain of bacteria may be any strain that expresses at least one amino acid sequence set forth in SEQ ID NOs: 1-5 or 7-14. The strain of bacteria may be any strain that comprises at least one nucleic acid sequence set forth in SEQ ID NOs: 6, 15 or any other sequence disclosed herein. Exemplary Propionibacterium bacterium strains for use in the methods and compositions provided herein include, but are not limited to, HL037PA1, HL078PA1, HL053PA2, HL082PA1, HL086PA1, HL092PA1, HL110PA1, HL110PA2, HL030PA2, HL030PA1, HL063PA2, PV-66, Type IA2 P.acn17, HL097PA1, PRP-38, 5_U_42AFAA, HL082PA2 (e.g., P. acnes strains). Exemplary Propionibacterium bacterium strains for use in the methods and compositions provided herein also include, but are not limited to HL083PV1 or HGH0353 (e.g., P. avidum strains). The strain may be any strain in FIGS. 1-7 disclosed herein.

The compositions provided herein may include at least one, at least two, at least three, at least four, at least five, at least six, at least seven, at least eight, at least nine, or at least ten of the bacterial strains provided herein. The compositions provided herein may include at least one, at least two, at least three, at least four, at least five, at least six, at least seven, at least eight, at least nine, or at least ten of any bacterial strain that comprises Locus 4, or at least one bacterial strain that encodes for at least one peptide with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with the nucleic acid sequence of Locus 4. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with any amino acid sequence set forth in SEQ ID NOs; 1-5 or 7-14, or any bacterial strain that produces agent X.

The compositions provided herein may include at least one, at least two, at least three, at least four, at least five, at least six, at least seven, at least eight, at least nine, or at least ten of any bacterial strain that comprises Locus 4, or at least one bacterial strain that comprises a nucleic acid sequence with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with the nucleic acid sequence of Locus 4. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with any nucleic acid sequence set forth in SEQ ID NOs; 6 or 15, or any other sequence disclosed herein.

The strain of bacteria may be any strain that produces or expresses a peptide with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with the nucleic acid sequence of Locus 4. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with at least one amino acid sequence set forth in SEQ ID NOs: 1-5 or 7-14. As used herein, any strain of bacteria that that produces or encodes a peptide encoded in SEQ ID Nos: 1-5 or 7-14 includes any bacterial strain that produces or encodes a peptide with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with the nucleic acid sequence of Locus 4. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with at least one amino acid sequence set forth in SEQ ID NOs: 1-5 or 7-14. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with at least one nucleic acid sequence set forth in herein (e.g., SEQ ID NOs: 6 or 15).

The Propionibacterium (recently renamed to Cutibacterium) bacterium may be P. acnes. The Propionibacterium bacterium may be P. avidum. In some embodiments, the disease is a caused by Gram-positive bacteria. The Gram-positive bacteria may be Staphylococcus, such as Methicillin-resistant Staphylococcus aureus (MRSA). The Gram-positive bacteria may be Enterococcus, such as Vancomycin-resistant Enterococcus (VRE). The Gram-positive bacteria may be Streptococcus, such as Group A Streptococcus (GAS). The Gram-positive bacteria may be C. difficle. The Gram-positive bacteria may be Streptococcus, such as Group A Streptococcus (GAS). The Gram-positive bacteria may be P. acnes, P. avidum, P. granulosum, or P. humerusii. In some embodiments, the disease may be a skin disease. In some embodiments, the disease may be a skin disease or any other disease associated with inflammation (e.g., atopic dermatitis or acne).

In some aspects, provided herein are methods of treating or preventing an infection caused by Gram-positive bacteria in a subject in need thereof by administering a composition comprising a strain of bacteria comprising Locus 4 (e.g., a P. acnes or P. avidum strain comprising Locus 4), a composition comprising a strain of bacteria that expresses any one of SEQ ID NOs: 1-5 or 7-14, a composition comprising a strain of bacteria that comprises an nucleic acid set forth in SEQ ID NO: 6, 15 or any nucleic acid disclosed herein, or a composition comprising agent X to the subject. In some aspects, provided herein are methods of treating or preventing an infection caused by Gram-positive bacteria in a subject in need thereof by administering a composition comprising a Propionibacterium bacterium that expresses agent X to the subject.

In some aspects, provided herein are methods of reducing the levels of a Gram-positive bacteria in a subject or on a subject's skin in need thereof by administering a composition comprising agent X to the subject. In some aspects, provided herein are reducing the levels of a Gram-positive bacteria in or on a subject in need thereof by administering a composition comprising a Propionibacterium bacterium that produces agent X to the subject, a strain of bacteria comprising Locus 4 (e.g., a P. acnes or P. avidum strain comprising Locus 4), a composition comprising a strain of bacteria that expresses any one of SEQ ID NOs: 1-5 or 7-14, a composition comprising a strain of bacteria that comprises an nucleic acid set forth in SEQ ID NO: 6, 15 or any nucleic acid disclosed herein, or a composition comprising agent X to the subject. The Propionibacterium bacterium may be P. acnes. The Propionibacterium bacterium may be P. avidum. The Gram positive bacteria may be Staphylococcus, such as Methicillin-resistant Staphylococcus aureus (MRSA). The Gram positive bacteria may be Enterococcus, such as Vancomycin-15 resistant Enterococcus (VRE). The Gram positive bacteria is Streptococcus, such as Group A Streptococcus (GAS). In some embodiments, the subject has a skin disease associated with inflammation (e.g., atopic dermatitis or acne). In some embodiments, the Gram-positive bacteria is Clostridium difficile.

Agent X may be a microbial peptide encoded by Locus 4. Agent X may comprise a least one peptide with an amino acid sequence of any one of SEQ ID NOs: 1-5 or 7-14 (e.g., agent X may comprise at least one peptide with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology to any one of SEQ ID NOs: 1-5 or 7-14). Agent X may comprise a peptide encoded by a nucleic acid sequence of any one of SEQ ID NOs: 6, 15 or any other sequence disclosed herein (e.g., agent X may comprise at least one nucleic acid with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology to any one of SEQ ID NOs: 6, 15, or any other sequence disclosed herein). Agent X may be a lantibiotic. Agent X may be expressed by Propionibacterium bacterium. The Propionibacterium bacterium may be P. acnes (e.g., RT8, RT3, RT1, and RT5 P. acnes strains or P. acnes belongs to the IB-1, IB2, and IC clades). The Propionibacterium bacterium may be P. avidum.

In some embodiments, the composition further comprises an antibiotic, In some embodiments, the composition is formulated for topical delivery. In other embodiments, the composition is formulated for oral delivery. In some embodiments, the subject is human. The methods provided herein include methods of administering separate compositions, each composition comprises at least one, at least two, at least three, at least four, at least five, at least six, at least seven, at least eight, at least nine, or at least ten of the bacterial strains provided herein. Each composition to be administered may include at least one, at least two, at least three, at least four, at least five, at least six, at least seven, at least eight, at least nine, or at least ten of any bacterial strain that comprises Locus 4, encodes for at least one peptide with at least 50% homology to any amino acid sequence set forth in SEQ ID Nos: 1-5 or 7-14, comprises homology with any nucleic acid sequence disclosed herein, or any bacterial strain that produces agent X. The compositions may be administered concurrently or sequentially. The compositions may be administered conjointly. Locus 4 may comprise at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with SEQ ID NO: 6, 15 or any other nucleic acid sequence disclosed herein.

In certain embodiments, agents and/or compositions of the invention may be used alone or conjointly administered with another type of therapeutic agent and/or a second composition disclosed herein. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In some embodiments, the methods provided herein include further conjoint administration of a composition (e.g., a pharmaceutical or cosmetic composition) comprising iron and/or cobalt to the subject. In some embodiments, the subject that receives conjoint administration has a skin disease (e.g., inflammatory skin disease, such as acne, rosacea, psoriasis, or Porphyria Cutanea Tarda (PCT)).

In some aspects, provided herein are methods and compositions related to treating or preventing a skin disease (e.g., inflammatory skin disease, such as acne, psoriasis, rosacea, or Porphyria Cutanea Tarda (PCT)), preventing and/or slowing skin aging (e.g., preventing the formation of wrinkles), and maintaining healthy skin by administering to the subject a composition disclosed herein. The compositions disclosed herein may be administered conjointly with a composition comprising iron and/or cobalt.

In some embodiments, the subject is in need of reducing the level porphyrins (e.g., bacterially derived porphyrins) on the skin of a subject by administering (e.g., a subject with symptoms of skin aging, or a subject with a skin condition, such as a skin disease associated with inflammation, acne, psoriasis, rosacea, PCT, or any other skin disease disclosed herein) a composition disclosed herein to the subject. The porphyrins may be produced by P. acnes (e.g., acne-associated strains of P. acnes). The porphyrins may be produced by P. granulosum, P. avidum, or P. humerusii (e.g., disease-associated strains of P. granulosum, P. avidum, or P. humerusii). The composition may be administered. conjointly with a compositions comprising iron and/or cobalt.

### BRIEF DESCRIPTION OF THE FIGURES

FIG. 1 shows the Locus 4 region of P. acnes encodes Pilosebin L4, a type III lantibiotic. Genes found in locus 4 are colored by their corresponding KEGG Orthology assignments. HL030PA2 strain of P. acnes is used as a representative, with gene numbers corresponding to locus tag ID (HMPREF9602_0XXXX). The type III lantibiotic gene cluster detected by BAGEL3 is underlined.

FIG. 2A-FIG. 2B shows a phylogenetic tree highlighting taxa within the Actinomycetales order that encode for the lantibiotic from locus 4. (FIG. 2A) Red taxa encode for full locus 4 with lantibiotic gene cluster, while blue taxa encode for partial locus 4 lacking the lantibiotic gene cluster. The lantibiotic gene cluster is restricted to P. acnes and P. avidum lineages. All nodes have Bayesian posterior probabilities greater than 0.9. Branches with Maximum Likelihood bootstrap support over 70% are thickened. Bifidobacterium bifidum was used as the outgroup. (FIG. 2B) Cladogram of the phylogenetic reconstruction of part A. Green branches indicate lineages that have retained locus 4. P. acnes clades and ribotypes are indicated above branches.

FIG. 3 shows that Pilosebin L4 producers can inhibit a broad range of Gram-positive bacteria, but not Gram-negative bacteria. Inhibitory activity of PL4 produced from 10 P. acnes strains (RT1, RT8, RT3, and RT5 strains) and 1 P. avidum strain (shown in columns) against 17 P. acnes strains , 3 other Propionibacterium species, and multiple Gram-positive species (shown in rows) were measured. The sizes of the inhibition zones (mm) are colored as a gradient with black boxes indicating highest inhibition, and white boxes indicating no inhibition. P. acnes strain HL037PA1, which does not encode PilosebinL4, is shown as a negative control.

FIG. 4 shows P. acnes strains containing L4 locus inhibit the growth of PilosebinL4-negative P. acnes strains (HL030PA1 is shown here as an example) and other Gram+bacteria. Two Staphyloccocus aureus strains, S. aureus 4330 (MRSA) and S. aureus 29213 (MSSA) are shown here as examples.

FIG. 5 shows P. acnes strains containing L4 locus inhibit the growth of PilosebinL4-negative P. acnes strains and other Gram+bacteria, including Staphyloccocus aureus, Clostridium difficile, and Bacillus subtilis.

FIG. 6 shows that Pilosebin L4 is regulated by cell density. P. acnes. HL030PA2 cultures of decreasing cell densities were used to measure RNA expression levels of the major lantibiotic modification enzyme (LanKC) and the histidine kinase regulatory gene (HISK) in the lantibiotic gene cluster. Expression of both genes were normalized to the single copy housekeeping gene pak and shown as relative expression against undiluted cultures. *p<0.05, **p<0.01, ***p<0.005, ****p<0.0005.

FIG. 7 shows bacterial species expressing Pilosebin L4 can outcompete PilosebinL4-negative cells in a microbial community. Mock communities of PilosebinL4 expressing P. acnes and P. avidum (blue columns) and L4-negative P. acnes strains (grey columns) were mixed in various ratios and combinations (“input” communities). Strain composition after 48 hours of growth was measured (“output” communities) using qPCR and strain-specific primers. *p<0.05, ***p<0.0005, ****p<0.0001.

FIG. 8 shows the changes in expression levels of Locus 4 genes between pure culture and co-culture based on transcriptomic analysis. Upregulation of several genes in a co-culture bacterial community compared to pure culture was observed, suggesting these genes play a role in Pilosebin L4 regulation. Right column co-culture, Left column pure culture.

FIG. 9 shows Actinomycetales species harboring partial locus 4 genes. Remnants of locus 4 were detected in 7 species, based on BLASTn searches against full, partial, and draft bacterial genomes on IMG. The full locus 4 with the lantibiotic operon is represented by P. acnes HL030PA2. Genes are colored according to their KO assignments. Gene numbers corresponds to locus tag IDs: P. acnes HL030PA2, HMPREF9602_0XXXX; P. acidifaciens DSM 21887, K336DRAFT_0XXXX; Streptomyces sp. ATexAB-D23, B082DRAFT_0XXXX; Streptomyces sp. 303MFCol5.2, H294DRAFT_0XXXX; K. cheerisanensis KCTC 2395, KCH_XXXXX; K. setae KM-6054, KSE_XXXXX; Kitasatospora sp. SolWspMP-SS2h, K353DRAFT_0XXXX; K. mediocidica KCTC 9733, BS80DRAFT_0XXXX.

FIG. 10 shows Locus 4 is integrated in the same genetic loci in P. acnes and P. avidum. The genetic loci of locus 4 is displayed with genes flanked on both ends. Locus 4 is not drawn to scale relative to other genes. Genes are colored by their KO assignments.

### DETAILED DESCRIPTION

Applicant has identified a lantibiotic (bacteriocin) that is uniquely expressed in certain strains of P. acnes and a related skin bacterium, Propionibacterium avidum. This lantibiotic is encoded in a unique genomic region named Locus 4 (Tomida et al., 2013). Lantibiotics are a type of bacteriocin that can kill other cells in the bacterial community and thus change the population and dynamics of the microbiome. The lantibiotic identified in this invention, which is named Pilosebin L4 or agent X, can inhibit the growth of a broad range of Gram-positive bacteria, including common skin bacterial species as well as important clinical pathogens such as Methicillin-resistant Staphylococcus aureus (MRSA), Vancomycin-resistant Enterococcus (VRE), Group A Streptococcus (GAS), and Clostridium difficile. The Gram-positive bacteria may be P. acnes, P. avidum, P. granulosum, or P. humerusii (disease associated strains of P. acnes, P. avidum, P. granulosum, or P. humerusii). Applicant has demonstrated that P. acnes and P. avidum strains that contain Locus 4 and express Pilosebin L4 outcompete other bacterial strains that do not express the lantibiotic and become the dominant members of the community.

Provided herein are methods and compositions useful in the treatment of infections caused by Gram-positive bacteria. In some aspects, provided herein are methods of treating or preventing a disease (e.g., a skin disease, a disease of the GI tract, or any other disease) in a subject in need thereof by administering a composition comprising agent X to the subject or administering a composition comprising a Propionibacterium bacterium that expresses agent X to the subject.

In some aspects, provided herein are methods of treating or preventing an infection caused by Gram-positive bacteria in a subject in need thereof by administering a composition comprising agent X to the subject. In some aspects, provided herein are methods of treating or preventing an infection caused by Gram-positive bacteria in a subject in need thereof by administering a composition comprising a Propionibacterium bacterium that produces agent X, a composition comprising a strain of bacteria that expresses an amino acid of any one of SEQ ID NOs: 1-5 or 7-14, a composition comprising a strain of bacteria that comprises a nucleic acid of any one of SEQ ID NOs: 6, 15, or any other nucleic acid sequence disclosed herein, or a Propionibacterium bacterium strain that comprises Locus 4 to the subject.

In some aspects, provided herein are methods of reducing the levels of a Gram-positive bacteria in the subject or on the skin of a subject in need thereof by administering a composition comprising agent X, a composition comprising a strain of bacteria that expresses at least one amino acid set forth SEQ ID NOs: 1-5 or 7-14, a composition comprising a strain of bacteria that comprises a nucleic acid of any one of SEQ ID NOs: 6, 15, or any other nucleic acid sequence disclosed herein and/or a Propionibacterium bacterium strain that comprises Locus 4 to the subject. The Gram-positive bacteria may be in or on any site of the body, including, but not limited to, the skin, gastrointestinal tract, mouth, ear, blood, lung, surgical sites, urinary tract, or any other organ of the body.

Similarly, “infection” as used herein, can refer to an infection on any part of the body, including, but not limited to, the skin, gastrointestinal tract, mouth, ear, blood, lung, surgical sites, urinary tract, or any other organ of the body. In some aspects, provided herein are reducing the levels of a Gram-positive bacteria in or on the skin of a subject in need thereof by administering a composition comprising a Propionibacterium bacterium that expresses agent X and/or a Propionibacterium bacterium strain that comprises Locus 4 to the subject.

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of acne includes, for example, reducing the number of detectable acne lesions in a population of patients receiving a prophylactic treatment relative to an untreated control population, and/or delaying the appearance of detectable lesions in a treated population versus an untreated control population, e.g., by a statistically and/or clinically significant amount.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “ribotype” refers to strains of P. acnes. The ribotyped strains were characterized as in Fitz-Gibbon et al. (J. Investigative Dermatology 133:2152-60 (2013)).

The term “subject” refers to a mammal, including, but not limited to, a human or non-human mammal, such as a bovine, equine, canine, ovine, or feline.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### Therapeutic Methods

Provided herein are methods and compositions useful in the treatment of infections caused by Gram-positive bacteria. In some aspects, provided herein are methods of treating or preventing a disease (e.g., a skin disease or any disease disclosed herein) in a subject in need thereof by administering a composition comprising agent X to the subject or administering a composition comprising a Propionibacterium bacterium that produces agent X, a strain of bacteria comprising Locus 4 (e.g., a P. acnes or P. avidum strain comprising Locus 4), a composition comprising a strain of bacteria that comprises a nucleic acid of any one of SEQ ID NOs: 6, 15, or any other nucleic acid sequence disclosed herein, or a composition comprising a strain of bacteria that expresses any one of SEQ ID NOs: 1-5 or 7-14 to the subject. The infection caused by Gram-positive bacteria may be in or on any site of the body, including, but not limited to, the skin, gastrointestinal tract, mouth, ear, blood, lung, surgical sites, urinary tract, or any other organ of the body.

In some aspects, provided herein are methods of treating or preventing an infection caused by Gram-positive bacteria in a subject in need thereof by administering a composition comprising agent X, a strain of bacteria comprising Locus 4 (e.g., a P. acnes or P. avidum strain comprising Locus 4), a composition comprising a strain of bacteria that comprises a nucleic acid of any one of SEQ ID NOs: 6, 15, or any other nucleic acid sequence disclosed herein, or a composition comprising a strain of bacteria that expresses any one of SEQ ID NOs: 1-5 or 7-14 to the subject. In some aspects, provided herein are methods of treating or preventing an infection caused by Gram-positive bacteria in a subject in need thereof by administering a composition comprising a Propionibacterium bacterium that produces agent X, a strain of bacteria comprising Locus 4 (e.g., a P. acnes or P. avidum strain comprising Locus 4), a composition comprising a strain of bacteria that comprises a nucleic acid of any one of SEQ ID NOs: 6, 15, or any other nucleic acid sequence disclosed herein, or a composition comprising a strain of bacteria that expresses any one of SEQ ID NOs: 1-5 or 7-14 to the subject.

In some aspects, provided herein are methods of reducing the levels of a Gram-positive bacteria in (e.g., in the GI tract) or on the skin of a subject in need thereof by administering a composition comprising agent X and/or a Propionibacterium bacterium strain that comprises Locus 4 to the subject. In some aspects, provided herein are reducing the levels of a Gram-positive bacteria in (e.g., in the GI tract) or on the skin of a subject in need thereof by administering a composition comprising a Propionibacterium bacterium that produces agent X and/or a Propionibacterium bacterium strain that comprises Locus 4 to the subject.

In some embodiments, the skin disease is a caused by Gram-positive bacteria. The Gram-positive bacteria may be any Gram-positive bacteria that is pathological. The Gram-positive bacteria may be Staphylococcus, such as Methicillin-resistant Staphylococcus aureus (MRSA). The Gram-positive bacteria may be Enterococcus, such as Vancomycin-resistant Enterococcus (VRE). The Gram-positive bacteria is Streptococcus, such as Group A Streptococcus (GAS). The Gram-positive bacteria may be P. acnes, P. avidum, P. granulosum, or P. humerusii. In some embodiments, the disease is a skin disease. In some embodiments, the disease of the gastrointestinal tract. In some embodiments, the disease of the gastrointestinal tract. In some embodiments, the disease of the genitalia. In some embodiments, the disease is a skin disease associated with inflammation (e.g., atopic dermatitis or acne, such as acne caused by acne-causing strains of P. acnes).

The Propionibacterium bacterium may be P. acnes (e.g., RT8, RT1, RT5, or RT3 P. acnes or P. acnes that belongs to the IB-1, IB-2, or IC clades). The Propionibacterium bacterium may be P. avidum. The Gram positive bacteria may be Staphylococcus, such as Methicillin-resistant Staphylococcus aureus (MRSA). The Gram positive bacteria may be Enterococcus, such as Vancomycin-resistant Enterococcus (VRE). The Gram positive bacteria may be Streptococcus, such as Group A Streptococcus (GAS). The Gram-positive bacteria may be P. acnes, P. avidum, P. granulosum, or P. humerusii. In some embodiments, the subject has a skin disease associated with inflammation (e.g., atopic dermatitis or acne). In some embodiments, the Gram-positive bacteria is Clostridium difficile. In some embodiments, the subject is afflicted with or suffering from Methicillin-resistant Staphylococcus aureus (MRSA), Vancomycin-resistant Enterococcus (VRE), Group A Streptococcus (GAS), or Clostridium difficile.

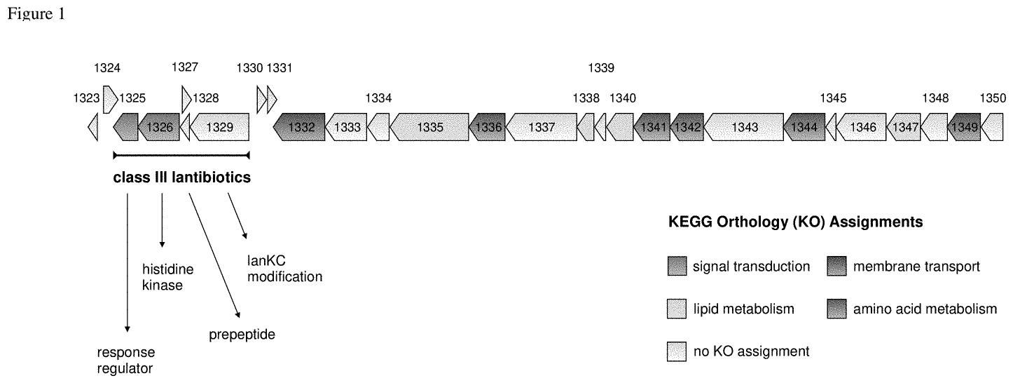

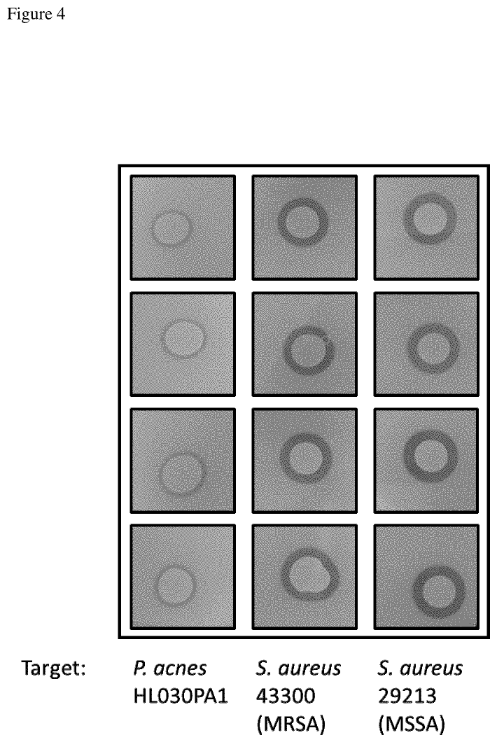

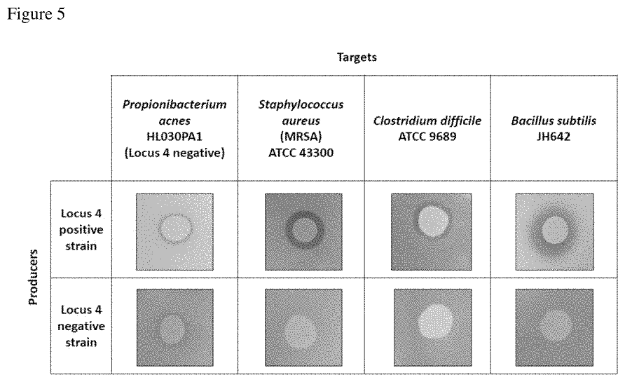

Agent X may be a lantibiotic, e.g., a class III lantibiotic. Agent X may be expressed by Propionibacterium bacterium. Agent X may comprise a peptide (e.g., at least one, at least two, at least three, at least four, at least five, at least six, or at least seven) with an amino acid sequence with at least 50%, at least 80%, at least 90%, at least 95%, at least 99%, or 100% homology with any one of SEQ ID NOs: 1-5 or 7-14. The Propionibacterium bacterium may be P. acnes (e.g., RT8, RT1, RT5, RT3 P. acnes strains or P. acnes that belongs to the IB-1, IB-2, or IC clades). The Propionibacterium bacterium may be any P. acnes highlighted in FIGS. 1-7. The Propionibacterium bacterium may be P. acnes HL030PA2 or P. acnes HL086PA1. The Propionibacterium bacterium may be any Propionibacterium that has retained Locus 4. The Propionibacterium bacterium may be P. avidum (e.g., P. avidum strains HGH0353 and HL083PV1 strains). The Propionibacterium bacterium may be any P. acnes or P. avidum strains that produce agent X.

Exemplary Propionibacterium acnes strain HL030PA2--proteins encoded by
lantibiotic region of locus 4
HMPREF9602_01325-response regulator receiver domain protein
MTTRILLAEDIKLVAEAFEALLSVEPEFEVVARVARGDDLVTSATRLFPDVILADID
MPGMTGIEATRMLRDKGYRGRIILLTALPGSGHLHAAMAAGADGYLLKSITAPSLI
NAIRAVCNGQSAIDPNVAAVAMRKGPSPLNDRETEILRLVADGSSTAQIASRLYLS
KGTVRNYLSTAMSKLDADSRTAAVTRAREEGWL (SEQ ID NO: 1)
HMPREF9602_01326-histidine kinase
MVRTRPVLDSPGRPLIASGVVLAVLLIIAVMTMVLQRDPTLTPGRRILGIVLSTVQA
VGFMWLQTRMVRHGKGSRNMRATLGRVGFWATWGLVAVGVVNLWLTNSWTLF
GASVATPLLLLPGVWSVVATLVTLIVGFSDLVVTHTSPLTSATVVLVTIAVSVNLY
AFTKLSTALVELRSSQEEIARLRVDAERHRISRDLHDIIGRTLVATSMRQQAALHLV
DRDPQRAKEQLDAAHDAITEGQHQLRSIIQAEMSTSLPDEISDATFLCDRLHIDFRM
DDRGRPHKPFDSAAAAALREGITNMLKHSAAGYCHVAISPDLLTVTNDGCPQSPRP
STTPGTGIDHLRSQIEALGGSVTTSRPSRGTFRLRCSFPTRGASTTTPATEGGR (SEQ
ID NO: 2)
HMPREF9602_01327-hypothetical protein
MASTDILSPGVHRGETSHACQGVLAQHVFLDVWSLSVVNYRR (SEQ ID NO: 3)
HMPREF9602_01328-hypothetical protein
MSVEAMQSIEMDEENSASQTVCACYSFFSWSGCCVVSQAVEAS (SEQ ID NO: 4)
HMPREF9602_01329-hypothetical protein
MYQATWRDGTSVVVKEARHLSGLDAAGVDARVRLRHEYEALRRLDPYRAAPHPI
DLIETEDGSFLIMEFLDGMTVHQEMSRFHPLIGRRPHRLSMTDFSRWCREVEDRLR
NVTRVMAGCGIVHGDLHPANLIHVGHEVLVTDFESCSIDGVAVSSGIAAPWFQSDD
TIPDPATTDDLHTLLVDPTCGPALVLHPNLRALISQAAVEDMMGNPTGLPHAWNLE
DVTSELAKGIRASATPARADRLFPSDPYLFGHPGSEFGLMHGAAGIMAALAVTGYG
VDANHVTWMKDRLATRPTLLPGLANGIEGIALGLSLCGQNDMAASLLHQSGILTIT
TNGSTDLTLGTGMAGRACALQSLSQRLSSKSLAHAAYTLWEQLAVAVRNTDVAL
PNGLFSGWEGIGLSLLASPLPDRHDLARHSLRLALATTTIVDGALFSGEGQIRWPYL
GRGPAACGPLAARLDDDQTAKAVAQTCRCPLTESSGLHNGRAGLLLVLRQLVGD
QDDAVRRHLMRLSWSMDRREEGTLLLGDHGLRFSSDLATGSAGALLALSRDPWR
NMTRMLGICDDTCHGPLVTV (SEQ ID NO: 5)
Propionibacterium acnes HL030PA2--DNA sequence of HMPREF9602_01329 to
HMPREF9602_01325
CTACAGCCAGCCCTCCTCGCGTGCCCGCGTGACCGCCGCTGTGCGTGAGTCGGC
GTCGAGCTTCGACATCGCCGTCGACAGGTAGTTCCTGACGGTGCCCTTGCTGAG
GTACAGCCGCGAGGCGATCTGCGCGGTGGAGGAACCGTCGGCGACTAGGCGCA
GAATCTCGGTTTCCCGGTCGTTGAGGGGCGAGGGTCCCTTGCGCATGGCGACCG
CGGCGACGTTCGGGTCGATGGCCGACTGGCCGTTGCACACCGCCCGGATGGCG
TTGATGAGGGAGGGCGCGGTGATCGACTTGAGCAGGTACCCGTCGGCTCCGGC
AGCCATCGCGGCGTGCAGGTGCCCGCTTCCCGGCAGGGCGGTGAGCAGGATGA
TTCTGCCGCGGTATCCCTTGTCGCGAAGCATTCGGGTGGCCTCGATGCCGGTCA
TCCCGGGCATGTCGATGTCGGCCAGGATCACGTCGGGGAACAGGCGGGTGGCG
GATGTGACCAGGTCGTCGCCGCGTGCCACCCGTGCGACGACCTCGAACTCCGGT
TCCACCGAGAGCAGTGCCTCGAAGGCCTCTGCCACCAGTTTGATGTCCTCGGCG
AGGAGGATTCTCGTCGTCATCGTCCTCCTTCGGTCGCGGGTGTGGTCGTGGACG
CCCCCCGGGTCGGGAAGGAGCAGCGGAGCCGGAAGGTGCCCCGCGAGGGCCG
CGACGTCGTCACCGACCCACCGAGCGCCTCGATCTGGGACCGGAGATGGTCGA
TTCCGGTGCCCGGTGTGGTGGAGGGGCGAGGTGATTGCGGACAACCGTCGTTG
GTGACGGTCAGGAGGTCCGGGGAGATGGCGACGTGGCAGTAGCCCGCCGCGCT
GTGCTTGAGCATGTTCGTGATTCCCTCGCGCAGGGCCGCCGCCGCCGCCGAGTC
GAAGGGTTTGTGCGGTCTGCCGCGGTCATCCATGCGGAAGTCGATGTGGAGTC
GGTCGCACAGGAATGTGGCGTCGCTGATCTCGTCGGGGAGGCTGGTGCTCATCT
CGGCCTGGATGATGGAACGCAACTGGTGCTGCCCCTCGGTGATTGCGTCATGGG
CGGCGTCGAGCTGTTCCTTGGCCCGTTGCGGGTCGCGGTCGACGAGGTGGAGG
GCCGCCTGCTGGCGCATCGACGTGGCGACCAGGGTGCGACCGATGATGTCGTG
GAGGTCCCGCGATATCCGGTGACGCTCGGCGTCCACGCGCAGCCGGGCGATCT
CCTCCTGGGAGGAGCGGAGTTCAACGAGTGCCGTCGACAGCTTCGTGAATGCG
TAGAGGTTCACCGACACCGCGATGGTCACCAGCACGACCGTGGCTGAGGTCAG
TGGCGAGGTGTGGGTGACCACGAGGTCGGAGAAACCGACGATCAGGGTCACGA
GGGTGGCCACCACACTCCACACCCCTGGCAGGAGAAGGAGGGGCGTGGCGACG
GAGGCCCCGAAGAGGGTCCACGAGTTGGTCAGCCACAGATTGACGACTCCCAC
TGCGACGAGTCCCCACGTGGCCCAGAAACCGACCCTTCCAAGGGTCGCACGCA
TGTTGCGCGAACCCTTGCCGTGACGAACCATGCGGGTCTGCAGCCACATGAATC
CGACGGCCTGGACCGTGGACAGGACGATACCGAGAATGCGGCGTCCGGGGGTG
AGAGTGGGGTCGCGCTGAAGCACCATCGTCATCACCGCGATGATGAGCAGGAC
CGCCAGGACGACTCCGGAGGCGATGAGCGGTCTTCCGGGACTGTCCAGGACGG
GGCGTGTGCGTACCACGGGGGACAGGGTAGTCGACGGCGTCTCACACGATCAA
GATGCTTCCACCGCTTGGGAGACTACGCAGCATCCTGACCACGAGAAGAAGCT
GTAACAAGCGCAAACTGTCTGGCTGGCGCTGTTTTCCTCGTCCATCTCGATGGA
TTGCATGGCCTCAACGGACATTCTGTCTCCTGGTGTTCATCGTGGTGAAACGAG
CCACGCCTGCCAAGGCGTTTTGGCTCAGCATGTTTTCCTCGATGTTTGGAGTCTT
TCAGTTGTGAACTATCGGCGCTAGATCAAACTGTCACTAGTGGGCCATGACAGG
TGTCATCGCATATTCCGAGCATTCGTGTCATGTTCCGCCATGGGTCCCTGCTCAG
GGCGAGGAGGGCGCCGGCTGACCCCGTCGCGAGATCGGATGAGAAACGCAGG
CCATGATCACCCAGCAGCAATGTGCCTTCCTCGCGACGGTCCATCGACCACGAC
AGTCTCATGAGGTGGCGGCGAACGGCGTCGTCTTGGTCTCCCACGAGCTGGCG
GAGGACGAGTAGGAGACCTGCGCGGCCATTGTGGAGACCGGATGACTCAGTCA
GGGGACAACGACAGGTCTGTGCCACGGCCTTGGCCGTCTGGTCATCATCAAGC
CTGGCGGCGAGCGGGCCACATGCCGCGGGACCACGCCCCAGATATGGCCATCG
GATCTGTCCCTCGCCTGAGAAGAGCGCACCGTCAACAATCGTTGTCGTGGCCAG
GGCCAGTCGGAGTGAGTGGCGGGCGAGATCATGACGATCCGGCAGGGGAGAG
GCAAGCAGGGAGAGCCCGATCCCCTCCCAACCGGAGAACAGGCCGTTCGGCAA
GGCCACATCAGTGTTCCGCACAGCCACGGCGAGTTGTTCCCAGAGGGTATATGC
AGCGTGGGCGAGAGATTTCGATGACAGTCGTTGTGAGAGGGACTGCAGCGCGC
ACGCTCGGCCCGCCATACCAGTACCGAGGGTGAGGTCAGTCGATCCGTTCGTTG
TGATCGTGAGAATTCCGGACTGGTGGAGCAGGGATGCAGCCATGTCGTTCTGA
CCGCACAGGGAGAGACCCAGGGCAATTCCCTCGATTCCGTTGGCCAACCCCGG
CAGCAGGGTCGGGCGGGTGGCCAAGCGGTCCTTCATCCAGGTCACGTGATTCG
CGTCAACACCGTACCCAGTCACGGCAAGAGCAGCCATGATTCCGGCGGCGCCA
TGCATCAGGCCGAATTCTGAACCTGGATGCCCGAACAGGTAGGGGTCTGAGGG
AAAGAGGCGGTCGGCTCGTGCGGGAGTGGCTGATGCCCGGATCCCCTTGGCGA
GTTCGCTTGTCACGTCCTCGAGGTTCCAGGCATGTGGTAAACCAGTCGGATTTC
CCATCATGTCCTCGACTGCGGCCTGGCTGATGAGCGCGCGAAGATTTGGATGGA
GGACAAGCGCGGGGCCGCAAGTCGGATCCACGAGAAGAGTGTGGAGATCGTC
AGTCGTCGCCGGATCGGGAATCGTGTCGTCCGACTGGAACCAGGGAGCGGCGA
TCCCTGACGAGACGGCGACCCCGTCGATTGAGCAGGATTCAAAGTCAGTAACC
AAAACCTCATGGCCGACATGTATCAGATTCGCGGGATGAAGATCCCCGTGAAC
GATTCCGCACCCGGCCATGACCCTCGTGACATTCCTCAAGCGATCTTCGACCTC
TCGGCACCAGCGTGAAAAATCGGTCATCGATAGCCGATGAGGTCGCCTACCAA
TCAGAGGATGGAACCTCGACATCTCCTGGTGGACTGTCATTCCGTCAAGGAATT
CCATGATCAGGAACGAGCCGTCCTCGGTCTCAATGAGATCAATGGGATGGGGA
GCGGCCCGATAGGGGTCCAGTCTTCTCAGGGCCTCGTACTCGTGACGCAGCCGC
ACTCGGGCGTCGACACCCGCCGCATCAAGTCCGGACAGGTGCCGGGCCTCCTT
GACGACTACCGATGTGCCGTCTCTCCATGTTGCCTGATACAC(SEQ ID NO: 6)
Additional Exemplary Propionibacterium acnes lantibiotic region of locus 4
Response regulator, two-component system, NarL family
Strains (100% sequence identity in all these strains) and Locus tag:
HL030PA2 HMPREF9602_01325
HL053PA2 HMPREF9565_00684
HL063PA2 HMPREF9612_00736
HL078PA1 HMPREF9569_00974
HL082PA1 HMPREF9618_00908
HL086PA1 HMPREF9591_00696
HL092PA1 HMPREF9584_00678
HL110PA1 HMPREF9575_01636
HL110PA2 HMPREF9576_00295
Amino acid (aa) sequence:
MTTRILLAEDIKLVAEAFEALLSVEPEFEVVARVARGDDLVTSATRLFPDVILADID
MPGMTGIEATRMLRDKGYRGRIILLTALPGSGHLHAAMAAGADGYLLKSITAPSLI
NAIRAVCNGQSAIDPNVAAVAMRKGPSPLNDRETEILRLVADGSSTAQIASRLYLS
KGTVRNYLSTAMSKLDADSRTAAVTRAREEGWL (SEQ ID NO: 7)
Sensor histidine kinase, two-component system, NarL family
Strains (100% sequence identity in all these strains) and Locus tag:
HL030PA2 HMPREF9602_01326
HL053PA2 HMPREF9565_00683
HL063PA2 HMPREF9612_00735
HL078PA1 HMPREF9569_00975
HL082PA1 HMPREF9618_00909
HL086PA1 HMPREF9591_00695
HL092PA1 HMPREF9584_00679
HL110PA1 HMPREF9575_01637
HL110PA2 HMPREF9576_00296
Amino acid (aa) sequence:
MVRTRPVLDSPGRPLIASGVVLAVLLIIAVMTMVLQRDPTLTPGRRILGIVLSTVQA
VGFMWLQTRMVRHGKGSRNMRATLGRVGFWATWGLVAVGVVNLWLTNSWTLF
GASVATPLLLLPGVWSVVATLVTLIVGFSDLVVTHTSPLTSATVVLVTIAVSVNLY
AFTKLSTALVELRSSQEEIARLRVDAERHRISRDLHDIIGRTLVATSMRQQAALHLV
DRDPQRAKEQLDAAHDAITEGQHQLRSIIQAEMSTSLPDEISDATFLCDRLHIDFRM
DDRGRPHKPFDSAAAAALREGITNMLKHSAAGYCHVAISPDLLTVTNDGCPQSPRP
STTPGTGIDHLRSQIEALGGSVTTSRPSRGTFRLRCSFPTRGASTTTPATEGGR
(SEQ ID NO: 8)
Hypothetical Strains (100% sequence identity in all these strains) and Locus tag:
HL030PA2 HMPREF9602_01327
HL053PA2 HMPREF9565_00682
HL063PA2 HMPREF9612_00734
HL078PA1 HMPREF9569_00976
HL082PA1 HMPREF9618_00910
HL086PA1 HMPREF9591_00694
HL092PA1 HMPREF9584_00680
HL110PA1 HMPREF9575_01638
HL110PA2 HMPREF9576_00297
Amino acid (aa) sequence:
MASTDILSPGVHRGETSHACQGVLAQHVFLDVWSLSVVNYRR (SEQ ID NO: 9)
Hypothetical protein
Strains (100% sequence identity in all these strains) and Locus tag:
HL030PA2 HMPREF9602_01328
HL053PA2 HMPREF9565_00681
HL063PA2 HMPREF9612_00733
HL078PA1 HMPREF9569_00977
HL082PA1 HMPREF9618_00911
HL086PA1 HMPREF9591_00693
HL092PA1 HMPREF9584_00681
HL110PA1 HMPREF9575_01639
HL110PA2 HMPREF9576_00298
Amino acid (aa) sequence:
MSVEAMQSIEMDEENSASQTVCACYSFFSWSGCCVVSQAVEAS (SEQ ID NO: 10)
Hypothetical protein
1) Strains and Locus tag:
HL030PA2 HMPREF9602_01329
HL063PA2 HMPREF9612_00732
Amino acid (aa) sequence:
MYQATWRDGTSVVVKEARHLSGLDAAGVDARVRLRHEYEALRRLDPYRAAPHPI
DLIETEDGSFLIMEFLDGMTVHQEMSRFHPLIGRRPHRLSMTDFSRWCREVEDRLR
NVTRVMAGCGIVHGDLHPANLIHVGHEVLVTDFESCSIDGVAVSSGIAAPWFQSDD
TIPDPATTDDLHTLLVDPTCGPALVLHPNLRALISQAAVEDMMGNPTGLPHAWNLE
DVTSELAKGIRASATPARADRLFPSDPYLFGHPGSEFGLMHGAAGIMAALAVTGYG
VDANHVTWMKDRLATRPTLLPGLANGIEGIALGLSLCGQNDMAASLLHQSGILTIT
TNGSTDLTLGTGMAGRACALQSLSQRLSSKSLAHAAYTLWEQLAVAVRNTDVAL
PNGLFSGWEGIGLSLLASPLPDRHDLARHSLRLALATTTIVDGALFSGEGQIRWPYL
GRGPAACGPLAARLDDDQTAKAVAQTCRCPLTESSGLHNGRAGLLLVLRQLVGD
QDDAVRRHLMRLSWSMDRREEGTLLLGDHGLRFSSDLATGSAGALLALSRDPWR
NMTRMLGICDDTCHGPLVTV (SEQ ID NO: 11)
2) Strains and Locus tag: (99% identical to above sequence in HL030PA2 and
HL063PA2, 3 aa difference)
HL053PA2 HMPREF9565_00680
HL078PA1 HMPREF9569_00978
HL082PA1 HMPREF9591_00692
HL086PA1 HMPREF9618_00912
Amino acid (aa) sequence:
MYQATWRDGTSVVVKEARHLSGLDAAGVDARVRLRHEYEALRRLDPYRAAPHPI
DLIETEDGSFLIMEFLDGMTVHQEMSRFHPLISRRPHRLSMTDFSRWCREVEDRLRN
VTRVMAGCGIVHGDLHPANLIHVGHEVLVTDFESCSIDGVAVSSGIAAPWFQSDDT
IPDPATTDDLHTLLVDPTCGPALVPHPNLRALISQAAVEDMMGNPTGLPHAWNLE
DMTSELAKGIRASATPARADRLFPSDPYLFGHPGSEFGLMHGAAGIMAALAVTGY
GVDANHVTWMKDRLATRPTLLPGLANGIEGIALGLSLCGQNDMAASLLHQSGILTI
TTNGSTDLTLGTGMAGRACALQSLSQRLSSKSLAHAAYTLWEQLAVAVRNTDVA
LPNGLFSGWEGIGLSLLASPLPDRHDLARHSLRLALATTTIVDGALFSGEGQIRWPY
LGRGPAACGPLAARLDDDQTAKAVAQTCRCPLTESSGLHNGRAGLLLVLRQLVGD
QDDAVRRHLMRLSWSMDRREEGTLLLGDHGLRFSSDLATGSAGALLALSRDPWR
NMTRMLGICDDTCHGPLVTV (SEQ ID NO: 12)
3) Strains and Locus tag: (99% identical to above sequence in HL030PA2 and
HL063PA2, 4 aa difference)
HL092PA1 HMPREF9584_00682
HL110PA1 HMPREF9575_01640
Amino acid (aa) sequence:
MYQATWRDGTSVVVKEARHLSGLDAAGVDARVRLRHEYEALRRLDPYRAAPHPI
DLIETEDGSFLIMEFLDGMTVHQEMSRFHPLISRRPHRLSMTDFSRWCREVEDRLRN
VTRVMAGCGIVHGDLHPANLIHVGHEVLVTDFESCSIDGVAVSSGIAAPWFQSDDT
IPDPATTDDLHTLLVDPTCGPALVPHPNLRALISQAAVEDMMGNPTGLPHAWNLE
DMTSELAKGIRASATPARADRLFPSDPYLFGHPGSEFGLMHGAAGIMAALAVTGY
GVDANHVTWMKDRLATRPTLLPGLANGIEGIALGLSLCGQNDMAASLLHQSGILTI
TTNGSTDLTLGTGMAGRACALQSLSQRLSSKSLAHAAYTLWEQLAVAMRNTDVA
LPNGLFSGWEGIGLSLLASPLPDRHDLARHSLRLALATTTIVDGALFSGEGQIRWPY
LGRGPAACGPLAARLDDDQTAKAVAQTCRCPLTESSGLHNGRAGLLLVLRQLVGD
QDDAVRRHLMRLSWSMDRREEGTLLLGDHGLRFSSDLATGSAGALLALSRDPWR
NMTRMLGICDDTCHGPLVTV (SEQ ID NO: 13)
4) Strains and Locus tag: (99% identical to above sequence in HL030PA2 and
HL063PA2, 4 aa difference)
HL110PA2 HMPREF9576_00299
Amino acid (aa) sequence:
MYQATWRDGTSVVVKEARHLSGLDAAGVDARVRLRHEYEALRRLDPYRAAPHPI
DLIETEDGSFLIMEFLDGMTVHQEMSRFHPLISRRPHRLSMTDFSRWCREVEDRLRN
VTRVMAGCGIVHGDLHPANLIHVGHEVLVTDFESCSIDGVAVSSGIAAPWFQSDDT
IPDPATTDDLHTLLVDPTCGPALVPHPNLRALISQAAVEDMMGNPTSLPHAWNLED
MTSELAKGIRASATPARADRLFPSDPYLFGHPGSEFGLMHGAAGIMAALAVTGYG
VDANHVTWMKDRLATRPTLLPGLANGIEGIALGLSLCGQNDMAASLLHQSGILTIT
TNGSTDLTLGTGMAGRACALQSLSQRLSSKSLAHAAYTLWEQLAVAVRNTDVAL
PNGLFSGWEGIGLSLLASPLPDRHDLARHSLRLALATTTIVDGALFSGEGQIRWPYL
GRGPAACGPLAARLDDDQTAKAVAQTCRCPLTESSGLHNGRAGLLLVLRQLVGD
QDDAVRRHLMRLSWSMDRREEGTLLLGDHGLRFSSDLATGSAGALLALSRDPWR
NMTRMLGICDDTCHGPLVTV (SEQ ID NO: 14)
Propionibacterium acnes HL030PA2--DNA sequence of HMPREF9602_01329 to
HMPREF9602_01325
CTACAGCCAGCCCTCCTCGCGTGCCCGCGTGACCGCCGCTGTGCGTGAGTCGGC
GTCGAGCTTCGACATCGCCGTCGACAGGTAGTTCCTGACGGTGCCCTTGCTGAG
GTACAGCCGCGAGGCGATCTGCGCGGTGGAGGAACCGTCGGCGACTAGGCGCA
GAATCTCGGTTTCCCGGTCGTTGAGGGGCGAGGGTCCCTTGCGCATGGCGACCG
CGGCGACGTTCGGGTCGATGGCCGACTGGCCGTTGCACACCGCCCGGATGGCG
TTGATGAGGGAGGGCGCGGTGATCGACTTGAGCAGGTACCCGTCGGCTCCGGC
AGCCATCGCGGCGTGCAGGTGCCCGCTTCCCGGCAGGGCGGTGAGCAGGATGA
TTCTGCCGCGGTATCCCTTGTCGCGAAGCATTCGGGTGGCCTCGATGCCGGTCA
TCCCGGGCATGTCGATGTCGGCCAGGATCACGTCGGGGAACAGGCGGGTGGCG
GATGTGACCAGGTCGTCGCCGCGTGCCACCCGTGCGACGACCTCGAACTCCGGT
TCCACCGAGAGCAGTGCCTCGAAGGCCTCTGCCACCAGTTTGATGTCCTCGGCG
AGGAGGATTCTCGTCGTCATCGTCCTCCTTCGGTCGCGGGTGTGGTCGTGGACG
CCCCCCGGGTCGGGAAGGAGCAGCGGAGCCGGAAGGTGCCCCGCGAGGGCCG
CGACGTCGTCACCGACCCACCGAGCGCCTCGATCTGGGACCGGAGATGGTCGA
TTCCGGTGCCCGGTGTGGTGGAGGGGCGAGGTGATTGCGGACAACCGTCGTTG
GTGACGGTCAGGAGGTCCGGGGAGATGGCGACGTGGCAGTAGCCCGCCGCGCT
GTGCTTGAGCATGTTCGTGATTCCCTCGCGCAGGGCCGCCGCCGCCGCCGAGTC
GAAGGGTTTGTGCGGTCTGCCGCGGTCATCCATGCGGAAGTCGATGTGGAGTC
GGTCGCACAGGAATGTGGCGTCGCTGATCTCGTCGGGGAGGCTGGTGCTCATCT
CGGCCTGGATGATGGAACGCAACTGGTGCTGCCCCTCGGTGATTGCGTCATGGG
CGGCGTCGAGCTGTTCCTTGGCCCGTTGCGGGTCGCGGTCGACGAGGTGGAGG
GCCGCCTGCTGGCGCATCGACGTGGCGACCAGGGTGCGACCGATGATGTCGTG
GAGGTCCCGCGATATCCGGTGACGCTCGGCGTCCACGCGCAGCCGGGCGATCT
CCTCCTGGGAGGAGCGGAGTTCAACGAGTGCCGTCGACAGCTTCGTGAATGCG
TAGAGGTTCACCGACACCGCGATGGTCACCAGCACGACCGTGGCTGAGGTCAG
TGGCGAGGTGTGGGTGACCACGAGGTCGGAGAAACCGACGATCAGGGTCACGA
GGGTGGCCACCACACTCCACACCCCTGGCAGGAGAAGGAGGGGCGTGGCGACG
GAGGCCCCGAAGAGGGTCCACGAGTTGGTCAGCCACAGATTGACGACTCCCAC
TGCGACGAGTCCCCACGTGGCCCAGAAACCGACCCTTCCAAGGGTCGCACGCA
TGTTGCGCGAACCCTTGCCGTGACGAACCATGCGGGTCTGCAGCCACATGAATC
CGACGGCCTGGACCGTGGACAGGACGATACCGAGAATGCGGCGTCCGGGGGTG
AGAGTGGGGTCGCGCTGAAGCACCATCGTCATCACCGCGATGATGAGCAGGAC
CGCCAGGACGACTCCGGAGGCGATGAGCGGTCTTCCGGGACTGTCCAGGACGG
GGCGTGTGCGTACCACGGGGGACAGGGTAGTCGACGGCGTCTCACACGATCAA
GATGCTTCCACCGCTTGGGAGACTACGCAGCATCCTGACCACGAGAAGAAGCT
GTAACAAGCGCAAACTGTCTGGCTGGCGCTGTTTTCCTCGTCCATCTCGATGGA
TTGCATGGCCTCAACGGACATTCTGTCTCCTGGTGTTCATCGTGGTGAAACGAG
CCACGCCTGCCAAGGCGTTTTGGCTCAGCATGTTTTCCTCGATGTTTGGAGTCTT
TCAGTTGTGAACTATCGGCGCTAGATCAAACTGTCACTAGTGGGCCATGACAGG
TGTCATCGCATATTCCGAGCATTCGTGTCATGTTCCGCCATGGGTCCCTGCTCAG
GGCGAGGAGGGCGCCGGCTGACCCCGTCGCGAGATCGGATGAGAAACGCAGG
CCATGATCACCCAGCAGCAATGTGCCTTCCTCGCGACGGTCCATCGACCACGAC
AGTCTCATGAGGTGGCGGCGAACGGCGTCGTCTTGGTCTCCCACGAGCTGGCG
GAGGACGAGTAGGAGACCTGCGCGGCCATTGTGGAGACCGGATGACTCAGTCA
GGGGACAACGACAGGTCTGTGCCACGGCCTTGGCCGTCTGGTCATCATCAAGC
CTGGCGGCGAGCGGGCCACATGCCGCGGGACCACGCCCCAGATATGGCCATCG
GATCTGTCCCTCGCCTGAGAAGAGCGCACCGTCAACAATCGTTGTCGTGGCCAG
GGCCAGTCGGAGTGAGTGGCGGGCGAGATCATGACGATCCGGCAGGGGAGAG
GCAAGCAGGGAGAGCCCGATCCCCTCCCAACCGGAGAACAGGCCGTTCGGCAA
GGCCACATCAGTGTTCCGCACAGCCACGGCGAGTTGTTCCCAGAGGGTATATGC
AGCGTGGGCGAGAGATTTCGATGACAGTCGTTGTGAGAGGGACTGCAGCGCGC
ACGCTCGGCCCGCCATACCAGTACCGAGGGTGAGGTCAGTCGATCCGTTCGTTG
TGATCGTGAGAATTCCGGACTGGTGGAGCAGGGATGCAGCCATGTCGTTCTGA
CCGCACAGGGAGAGACCCAGGGCAATTCCCTCGATTCCGTTGGCCAACCCCGG
CAGCAGGGTCGGGCGGGTGGCCAAGCGGTCCTTCATCCAGGTCACGTGATTCG
CGTCAACACCGTACCCAGTCACGGCAAGAGCAGCCATGATTCCGGCGGCGCCA
TGCATCAGGCCGAATTCTGAACCTGGATGCCCGAACAGGTAGGGGTCTGAGGG
AAAGAGGCGGTCGGCTCGTGCGGGAGTGGCTGATGCCCGGATCCCCTTGGCGA
GTTCGCTTGTCACGTCCTCGAGGTTCCAGGCATGTGGTAAACCAGTCGGATTTC
CCATCATGTCCTCGACTGCGGCCTGGCTGATGAGCGCGCGAAGATTTGGATGGA
GGACAAGCGCGGGGCCGCAAGTCGGATCCACGAGAAGAGTGTGGAGATCGTC
AGTCGTCGCCGGATCGGGAATCGTGTCGTCCGACTGGAACCAGGGAGCGGCGA
TCCCTGACGAGACGGCGACCCCGTCGATTGAGCAGGATTCAAAGTCAGTAACC
AAAACCTCATGGCCGACATGTATCAGATTCGCGGGATGAAGATCCCCGTGAAC
GATTCCGCACCCGGCCATGACCCTCGTGACATTCCTCAAGCGATCTTCGACCTC
TCGGCACCAGCGTGAAAAATCGGTCATCGATAGCCGATGAGGTCGCCTACCAA
TCAGAGGATGGAACCTCGACATCTCCTGGTGGACTGTCATTCCGTCAAGGAATT
CCATGATCAGGAACGAGCCGTCCTCGGTCTCAATGAGATCAATGGGATGGGGA
GCGGCCCGATAGGGGTCCAGTCTTCTCAGGGCCTCGTACTCGTGACGCAGCCGC
ACTCGGGCGTCGACACCCGCCGCATCAAGTCCGGACAGGTGCCGGGCCTCCTT
GACGACTACCGATGTGCCGTCTCTCCATGTTGCCTGATACAC (SEQ ID NO: 15)

All of the referenced sequences are fully incorporated by reference in the form that was current on Jul. 22, 2020.

In some embodiments, the methods provided herein include further conjoint administration of a composition (e.g., a pharmaceutical or cosmetic composition) comprising iron and/or cobalt to the subject. In some embodiments, the subject that receives conjoint administration has a skin disease (e.g., inflammatory skin disease, such as acne, rosacea, psoriasis, or Porphyria Cutanea Tarda (PCT)). In some embodiments, the subject has symptoms of skin aging.

In some aspects, provided herein are methods of promoting vitamin B₁₂ biosynthesis in acne-associated strains of P. acnes on the skin of a subject by further administering a composition comprising cobalt and/or iron.

The compositions disclosed herein may further comprises one or more strains of P. acnes. The P. acnes strain may be RT1, RT2, RT3, or RT6 strain of P. acnes. The compositions may further comprise at least one phage against a strain of P. acnes (e.g., a phage that target a strain of P. acnes (e.g., RT4, RT5, RT7, RT8, RT9 or RT10). The compositions may further comprise P. granulosum, P. avidum, P. humerusii. Compositions disclosed herein may comprise an antibiotic (e.g., an antibiotic that does not target P. granulosum, P. avidum, P. humerusii, or an RT1, RT2, RT3, or RT6 strain of P. acnes).

The compositions disclosed herein may further comprise one or more strains of P. acnes (i.e., a strain associated with healthy skin, such as a RT1, RT2, RT3, or RT6 strain of P. acnes). The methods provided herein many further comprising administering one or more strains of P. acnes (i.e., a strain associated with healthy skin, such as a RT1, RT2, RT3, or RT6 strain of P. acnes). The compositions and method may further comprise at least one phage against a strain of P. acnes. The phage may target a strain of P. acnes that is associated with acne or an inflammatory skin disease, such as RT4, RT5, RT7, RT8, RT9 or RT10. The composition may comprise P. granulosum, P. avidum, P. humerusii. Compositions disclosed herein may comprise an antibiotic (e.g., an antibiotic that does not target P. granulosum, P. avidum, P. humerusii, or an RT1, RT2, RT3, or RT6 strain of P. acnes). Compositions may contain two or more, three or more, four or more, five or more, six or more, seven or more, eight or more, nine or more, or ten or more strains of P. acnes (e.g., an RT1, RT2, RT3, or RT6 strain of P. acnes), P. granulosum, P. avidum, and/or P. humerusii.

In some embodiments, the composition further comprises a phage against a strain of P. acnes (e.g., a RT4, RT5, RT7, RT8, RT9 or RT10 strain of P. acnes). In some embodiments, the composition comprises two or more (e.g., three or more, four or more, five or more, six or more, seven or more, eight or more, nine or more, or ten or more) phages against a strain of P. acnes. The type of phage that may be administered in a composition disclosed herein depends on the type of acne or skin disease, the medical history of an individual, or the symptoms of a subject with a skin disease. Non-limiting examples of phages include PHL111M01, PHL082M00, PHL060L00, PHL067M10, PHL071N05, PHL112N00, PHL037M02, PHL085N00, PHL115M02, PHL085M01, PHL114L00, PHL010M04, PHL066M04, PHL071N05, PHL113M01, PHL112N00, and PHL037M02. Information about P. acnes phages can be found in U.S. Patent Publication US20150086581A1, hereby incorporated in its entirety.

In some embodiments, the composition further comprises an antibiotic or the method further comprising administering and antibiotic to the subject (i.e., an antibiotic that does not target an agent X expressing bacterium, a bacterium that comprises Locus 4, or a bacterium described herein). In some embodiments, the subject is human.

In some embodiments, the methods provided herein include further conjoint administration of a composition (e.g., a pharmaceutical or cosmetic composition) comprising iron and/or cobalt to the subject. In some embodiments, the subject that is conjointly administered the compositions has a skin disease disclosed herein (e.g., inflammatory skin disease, such as acne, rosacea, psoriasis, or Porphyria Cutanea Tarda (PCT)).

In some aspects, provided herein are methods and compositions related to treating or preventing a skin disease disclosed herein, preventing and/or slowing skin aging (e.g., preventing the formation of wrinkles), and maintaining healthy skin by administering to the subject a composition disclosed herein. The skin disease may be acne, rosacea, psoriasis or PCT. The composition may be administered conjointly with a compositions comprising iron and/or cobalt.

In some embodiments, the subject is in need of reducing the level porphyrins (e.g., bacterially derived porphyrins) on the skin of a subject by administering (e.g., a subject with symptoms of skin aging, or a subject with any skin condition disclosed herein, such as a skin disease associated with inflammation, acne, rosacea, psoriasis, or PCT) a composition disclosed herein to the subject. The porphyrins may be produced by P. acnes (e.g., acne-associated strains of P. acnes). The porphyrins may be produced by P. granulosum, P. avidum, or P. humerusii (e.g., disease-associated strains of P. granulosum, P. avidum, or P. humerusii). The composition may be administered conjointly with a compositions comprising iron and/or cobalt.

The compositions disclosed herein may be heat treated, tyndallized and/or comprise supernatant-derived bacteria.

The compositions disclosed herein may be administered to a subject by any means known in the art, for example, the composition may be formulated for topical delivery. The formulation may be a liquid, gel, or cream. In some embodiments, the composition is formulated for oral delivery. The composition may be in the form of a pill, tablet, or capsule. In some embodiments, the subject may be a mammal (e.g., a human). In some embodiments, the composition is self-administered.

In general, the above methods directly act to reduce the amount of pathogenic or harmful bacteria in a subject. In some embodiments, this includes any such therapy that achieves the same goal of reducing the number of pathogenic or harmful organisms, when used in combination with the composition described herein, would lead to replacement of the pathogenic microflora involved in the diseased state with natural microflora enriched in a body site not afflicted with a disease, or less pathogenic species occupying the same ecological niche as the type causing a disease state. For example, a subject may undergo treatment with antibiotics or a composition comprising compounds to target and decrease the prevalence of pathogenic organisms, and subsequently be treated with a composition described herein.

Suitable antimicrobial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

Suitable antiviral agents include acyclovir, azidouridine, anismoycin, amantadine, bromovinyldeoxusidine, chlorovinyldeoxusidine, cytarabine, delavirdine, didanosine, deoxynojirimycin, dideoxycytidine, dideoxyinosine, dideoxynucleoside, desciclovir, deoxyacyclovir, efavirenz, enviroxime, fiacitabine, foscamet, fialuridine, fluorothymidine, floxuridine, ganciclovir, hypericin, idoxuridine, interferon, interleukin, isethionate, nevirapine, pentamidine, ribavirin, rimantadine, stavudine, sargramostin, suramin, trichosanthin, tribromothymidine, trichlorothymidine, trifluorothymidine, trisodium phosphomonoformate, vidarabine, zidoviridine, zalcitabine and 3-azido-3-deoxythymidine and analogs, derivatives, pharmaceutically acceptable salts, esters, prodrugs, and protected forms thereof.

Other suitable antiviral agents include 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-T-fluoroinosine, 2′,3′-dideoxy-2′-fluorothymidine, 2′,3′-dideoxy-2′-fluorocytosine, 2′3′-dideoxy-2′,3′-didehydro-T-fluorothymidine (Fd4T), 2′3′-dideoxy-T-beta-fluoroadenosine (F-ddA), 2′3′-dideoxy-2′-beta-fluoro-inosine (F-ddI), and 2′,3′-dideoxy-2′-beta-flurocytosine (F-ddC). In some embodiments, the antiviral agent is selected from trisodium phosphomonoformate, ganciclovir, trifluorothymidine, acyclovir, 3′-azido-3′-thymidine (AZT), dideoxyinosine (ddI), and idoxuridine and analogs, derivatives, pharmaceutically acceptable salts, esters, prodrugs, and protected forms thereof.

### Pharmaceutical Compositions

In some aspects, the invention relates to a composition (e.g., a pharmaceutical composition comprising agent X, a bacterium that produces agent X, a strain of bacteria comprising Locus 4 (e.g., a P. acnes or P. avidum strain comprising Locus 4), or a composition comprising a strain of bacteria that expresses any one of SEQ ID NOs: 1-5 or 7-14 disclosed herein. The compositions described herein (i.e., compositions comprising a strain of bacteria comprising Locus 4 or a strain of bacteria encoding for any one of SEQ ID NO: 1-5 or 7-14) may be formulated to promote the viability of live, replication-competent bacteria. Exemplary formulations are disclosed in WO2012077038, WO2011004375A1, WO2013188626, WO2011145737, and WO2010138522, each of which is incorporated in its entirety.

The strain of bacteria may be any strain which comprises Locus 4 (e.g., any strain with at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology to Locus 4). The strain of bacteria may be any strain that expresses at least one amino acid sequence set forth in SEQ ID NOs: 1-5 or 7-14. The strain of bacteria may be any strain that comprises a nucleic acid of any one of SEQ ID NOs: 6, 15, or any other nucleic acid sequence disclosed herein. Exemplary Propionibacterium bacterium strains for use in the methods and compositions provided herein include, but are not limited to, HL037PA1, HL078PA1, HL053PA2, HL082PA1, HL086PA1, HL092PA1, HL110PA1, HL110PA2, HL030PA2, HL030PA1, HL063PA2, PV-66, Type IA2 P.acn17, HL097PA1, PRP-38, 5_U_42AFAA, HL082PA2 (e.g., P. acnes strains). Exemplary Propionibacterium bacterium strains for use in the methods and compositions provided herein also include, but are not limited to HL083PV1 or HGH0353 (e.g., P. avidum strains). The compositions provided herein may include at least one, at least two, at least three, at least four, at least five, at least six, at least seven, at least eight, at least nine, or at least ten of the bacterial strains provided herein. The compositions provided herein may include at least one, at least two, at least three, at least four, at least five, at least six, at least seven, at least eight, at least nine, or at least ten of any bacterial strain that comprises Locus 4, any bacterial strain that encodes for at least one peptide with at least 50% homology to any amino acid sequence set forth in SEQ ID NOs; 1-5 or 7-14, or any bacterial strain that produces agent X. A strain of bacteria disclosed herein may be any strain which comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with Locus 4. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology with at least one amino acid sequence set forth in SEQ ID NOs: 1-5 or 7-14. The strain of bacteria may be any strain that comprises at least 50%, at least 60%, at least 70%, at least 80%, at least 90%, at least 91%, at least 92%, at least 93%, at least 94%, at least 95%, at least 96%, at least 97%, at least 98%, at least 99%, or 100% homology a nucleic acid of any one of SEQ ID NOs: 6, 15, or any other nucleic acid sequence disclosed herein.

The methods provided herein include methods of administering separate compositions. each compositions comprising at least one, at least two, at least three, at least four, at least five, at least six, at least seven, at least eight, at least nine, or at least ten of the bacterial strains provided herein. Each composition to be administered may include at least one, at least two, at least three, at least four, at least five, at least six, at least seven, at least eight, at least nine, or at least ten of any bacterial strain that comprises Locus 4, any bacterial strain that encodes for at least one peptide with at least 50% homology to any amino acid sequence set forth in SEQ ID NOs: 1-5 or 7-14, any bacterial strain that comprises a nucleic acid of any one of SEQ ID NOs: 6, 15, or any other nucleic acid sequence disclosed herein, or any bacterial strain that produces agent X. The compositions may be administered concurrently or sequentially. The compositions may be administered conjointly.

The pharmaceutical composition may be formulated for topical administration. The pharmaceutical composition may be a probiotic. The pharmaceutical compositions disclosed herein may be delivered by any suitable route of administration, including orally, buccally, sublingually, parenterally, and topically, as by powders, ointments, drops, liquids, gels, or creams. In certain embodiments, the pharmaceutical compositions are delivered generally (e.g., via oral or parenteral administration). In certain other embodiments, the pharmaceutical compositions are delivered locally through injection, micro needles, or patches.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

Exemplary identities of various constituents of the topical formulations of some embodiments of the present invention are described below.

### Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In some embodiments, formulations do not have methanol, ethanol, propanols, or butanol.

### Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-phosphate, methylbenzethonium chloride, sodium methyl cocoyl taurate, decyl glucoside, sodium cocoyl glutamate, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention.Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin. Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800. Suitable emollients or humectants for use in the formulations of the present invention include, but are not limited to, panthenol, acemannan, derivatives of Aloe vera, N-palmitoylethanolamine, N-acetylethanolamine, cetyl palmitate, glycerol (glycerin), PPG-stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl)palmitoylamino)-2-hydroxypropane. In addition, appropriate combinations and mixtures of any of these moisturizing agents and emollients may be used in accordance with the present invention.

### Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; bakuchiol, N-acetyl-L-cysteine, honokiol, magnolol, derivatives of Magnolia officinalis bark, chrysin, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols, D-ribose, N-acetyl-L-cysteine, apocynin, bixin, derivatives of Bixa orellana seeds, nicotinamide, acetyl zingerone, bakuchiol, tiliroside, extracts of Fragraria ananassa seed, fucoxanthin, creatine and its salts and esters, quercetin, luteolin, honokiol, magnolol, derivatives of Magnolia officinalis bark, tocopheryl acetate, sodium ascorbate/ascorbic acid, ascorbyl palmitate, propyl gallate, and chelating agents like EDTA (e.g., disodium EDTA), citric acid, and sodium citrate.

In some embodiments, the antioxidant or preservative comprises (3-(4-chlorophenoxy)-2-hydroxypropyl)carbamate.

In some embodiments, antioxidants or preservatives of the present invention may also function as a moisturizer or emollient, for example.

In addition, combinations or mixtures of these preservatives or anti-oxidants may also be used in the formulations of the present invention.

### Combination Agents

The composition can also contain any other agent that has a desired effect when applied topically to a subject. Suitable classes of active agents include, but are not limited to antibiotic agents (i.e., antibiotic agents that dot not target agent X producing organisms), antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

### Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate). For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

In some embodiments, the composition comprises a vitamin. Examples of vitamins include, but are not limited to, vitamins A, D, E, K, and combinations thereof. Vitamin analogues are also contemplated; for example, the vitamin D analogues calcipotriene or calcipotriol. In some embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

In some embodiments, the composition comprises a sunscreen. Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

### Exemplification

The human skin is host to a diverse community of bacterial species. Hosting a relatively pro-inflammatory bacterial population is thought to contribute to inflammatory skin diseases, while a non-inflammatory population is believed to maintain healthy skin. Lantibiotics are a type of bacteriocin that can cause great changes in the microbial community due to their ability to kill neighboring cells. In this study, a novel lantibiotic in Propionibacterium is identified, the dominant resident bacteria in sebaceous sites. The lantibiotic, which was named Pilosebin L4, can inhibit the growth of a broad range of Gram-positive bacteria, including those found on the skin as well as important clinical pathogens such as MRSA and VRE. Using mock bacterial communities, it was demonstrated that Propionibacterium strains that expressed Pilosebin L4 were able to out-compete those that do not express the lantibiotic and become the dominant members of the community. Colonization of a Propionibacterium strain expressing Pilosebin L4 can thus highly influence the microbiome composition and the host's susceptibility to skin diseases. Propionibacterium strains with pilosebin L4 can be further exploited as a probiotic to modulate the virulence property of the microbiome and to maintain skin health.

### Introduction

The human skin is host to a multitude of commensal bacterial communities. Propionibacterium species, primarily P. acnes, are the dominant genera at sebaceous sites (Fitz-Gibbon et al 2013, Grice et al 2009). Hosting a commensal bacterial flora is an important factor of skin health, and disruptions or shifts in skin microbial community are associated with a variety of inflammatory skin diseases, including acne vulgaris (acne)(Fitz-Gibbon 2013 and Barnard 2016). Acne is a chronic inflammatory disease of the pilosebaceous unit, characterized by papules and pustules arising on the affected skin. The cause of acne is multifactorial, characterized by hyperkeratiniation, increased sebum production, and pro-inflammatory bacteria in the affected region (Tanghetti 2013).

As the major colonizer of the pilosebaceous unit, along with its presence in both acneic and healthy skin, the role of P. acnes in acne pathogenesis has historically been debated. Recent studies have found that different lineages and strains of P. acnes have different genomic, transcriptomic, and metabolomic potential, suggesting that certain strains are more pathogenic, or pro-inflammatory than others (Brzuszkiewicz et al 2011, Fitz-Gibbon et al 2013, Johnson et al 2016, Kang et al 2015, McDowell et al 2012a, Tomida et al 2013). This suggests that having the health-associated P. acnes strains or community on the skin can help maintain a healthy skin status and reduce the incidence of inflammatory skin disease.

Bacteriocins are ribosomally synthesized antimicrobial peptides produced by a wide-range of bacteria. Lantibiotics (lanthionine containing antibiotics) are a type of bacteriocin produced by Gram-positive bacteria. They are characterized by their small sizes (<5 kDa), extensive post-translational modifications, and the presence of the amino acids lanthionine (Lan) and/or β-methyl-lanthionine (meLan) (Rea et al 2011b). The genes involved in lantibiotic biosynthesis are generally found on an operon, and encode for a precursor peptide and modification enzymes. Lantibiotic operons may also come with genes encoding for an exporter, protease, immunity proteins, and/or two-component regulatory system consisting of a histidine kinase receptor and a transcriptional response regulator (Chatterjee, 2005) (Dischinger et al 2014). Lantibiotic biosynthesis involves the translation of the prepeptide, followed by dehydration of serine and threonine residues to didehydroalanines (Dha) and didehydrobutyrines (Dhb), respectively. Subsequent intramolecular Michael addition of cysteine-SH groups to Dha/Dhb form the characteristic thioether crosslinks of Lan and MeLan (Goto et al 2010).

Lantibiotics are classified based on their biosynthetic genes: Class I are linear peptides with two modification enzymes LanB and LanC, a lantibiotic ABC transporter LanT, and a proteinase LanP that cleaves the leader peptides; Class II are generally more globular than Class I, and are modified by a single enzyme LanM, and processed and secreted by LanT; Class III are modified by LanKC (sometimes referred to as LabKC), while Class IV are modified by LanL (Alkhatib et al 2012, Dischinger et al 2014, Rea et al 2011b). Lantibiotics act by binding to lipid I or lipid II of the peptidoglycan cell wall to inhibit cell wall biosynthesis, or by forming pores in the cell membrane to kill the target bacteria (Brotz and Sahl 2000, Draper et al 2015). Only lantibiotics of Class I and Class II have shown antimicrobial activity (Goto et al 2010). Despite their lack of antimicrobial activity, many Class III lantibiotics have been well characterized (Krawczyk et al 2012, Krawczyk et al 2013, Voller et al 2012). To date, all Class III lantibiotics have been isolated from bacteria of the order Actinomycetales.

The production of bacteriocins such as lantibiotics by certain species/strains in a microbial community can have profound effects on community composition due to their ability to inhibit neighboring cells (Hawlena et al 2012, Perez-Gutierrez et al 2013). The production of bacteriocins by certain members of the microbiota has also been suggested to affect health and disease (Belda-Ferre et al 2012). There is only one reported case of bacteriocin characterization in P. acnes, identified from an oral isolate over 30 years ago (Fujimura and Nakamura 1978).

Previously, a lineage of P. acnes strains belonging to clade IB-1 and ribotyping scheme RT8 (Fitz-Gibbon et al 2013) was identified. Despite infrequently observed when present on the skin of acne patients, RT8 strains were in high relative abundance to almost 100% (Fitz-Gibbon et al 2013) (Barnard, 2016). In this study, a genome comparison and functional assays is combined to determine if these strains possessed genetic elements that allowed them to outcompete other bacterial species and strains in their community. A minor subset of P. acnes strains was discovered, including RT8 of clade IB-1, and some Propionibacterium avidum strains produced a novel lantibiotic that was named Pilosebin L4. Pilosebin L4 can kill a broad-range of Gram-positive bacteria, thus allowing the producer strains to dramatically change the microbial community composition. The implications for the presence of Pilosebin L4 in select propionibacteria and therapeutic applications are discussed.

### Materials & Methods

### Bacterial Strains and Culture Conditions

Bacterial strains, media, and culture conditions used in this study are summarized in Table 1. Culture medium A is a synthetic medium composed of (per litre): 12 g tryptone, 12 g yeast extract, 4 g glucose, 4 g KH2PO4, and 1 g MgSO4.7H2O. Propionibacterium granulosum, Propionibacterium humerusii, Staphylococcus epidermidis, P. avidum, and all P. acnes strains except for KPA171202 were previously isolated from human skin samples (Fitz-Gibbon et al 2013). Bacillus subtilis JH642 was kindly provided by Dr. Beth Lazazzera at UCLA. All other strains were purchased from sources listed in Table 1.

### Bacteriocin Genome Mining

The online tool BAGEL3 (van Heel et al 2013) was used to scan for bacteriocins in all publicly available genomes of P. acnes. FASTA files containing nucleotide sequences of all genomic scaffolds were used for analysis.

### KEGG Orthology (KO) Identities

The KEGG Automatic Annotation Server (KAAS) was used to determine the KO assignments of genes. The bi-directional best hit method was used on amino acid sequences, with the default prokaryotic genes data set.

### Phylogenetic Reconstruction

For the multi-gene phylogenetic reconstruction, amino acid sequences of the following 31 genes were selected and used, based on the Genomic Encyclopedia of Bacteria and Archaea (Wu et al 2009): dnaG, frr, infC, lepA, nusA, pgk, pnpA, rplA, rplB, rplC, rplD, rplE, rplF, rplK, rplL, rplM, rplP, rplS, rplT, rpmA, rpoA, rpoB, rpoD, rpsB, rpsC, rpsE, rpsI, rpsJ, rpsS, smpB, and tsf; all sequences were obtained from IMG (Markowitz et al 2012), except for P. avidum HL083PV1 and P. avidum HL307PV1, which were recently sequenced by the Applicant. Amino acid sequences were aligned using MUSCLE (Edgar 2004), and the individually aligned protein sequences were concatenated. A Bayesian phylogenetic tree was constructed in MrBayes v.3.2 (Ronquist et al 2012) using the amino acid model mixed+I+F to identify substitution models that best fit the data. The analysis was run with 25% burn-in for 200,000 generations. The average standard deviation of split frequencies was used as a convergence diagnostic. The Maximum Likelihood tree was constructed using PHYML (Guindon and Gascuel 2003) with 1,000 bootstrap replicates and the WAG+I+F (Whelan and Goldman 2001) amino acid substitution model.

### Inhibition Overlay Assay

Antimicrobial activity of the lantibiotic was detected using a soft agar overlay assay. Strains producing Pilosebin L4 were sub-cultured to OD595 of 1.0, spotted onto A-media plates, and incubated anaerobically at 37° C. for 48 h. Plates were then overlaid with soft agar media (0.7% agar +appropriate media as indicated in Table 1) containing target strains at OD595 of 1.0, and incubated anaerobically at 37° C. for 48 h for Propionibacterium species, or aerobically at 37° C. overnight for all other species. After incubation, overlays were examined for growth inhibition zones. Where observed, inhibition zones were measured from the inside edge (edge of the spotted producer strain) to the outer edge of the inhibition zone.

### RNA Extraction and Quantitative PCR Analysis

Fresh plate cultures of P. acnes HL030PA2 were harvested and sub-cultured in fresh BHI media to an OD595 of 1.0, and serially diluted in 1/5 dilutions up to 1/625. The dilutions were spread evenly on A-media plates, and incubated anaerobically at 37° C. for 48 h. Each plate, therefore, contained P. acnes HL030PA2 grown at various cell densities. Total RNA was extracted from each of the plates using RNAprotect Bacteria Reagent+RNeasy Mini Kit (Qiagen), following the protocol for “Disruption of Bacteria Grown on Solid Media” followed by the standard protocol for RNA purification. Potential DNA contaminants were removed using TURBO DNA-free DNase Treatment kit (Ambion). Total RNA was then converted to cDNA using SuperScript® III First-Strand Synthesis Kit (Invitrogen). qPCR was performed on the LightCycler® 480 System (Roche), with the LightCycler® 480 High Resolution Melting Dye (Roche) following the manufacturer's protocol. The following primer sets were used for the analysis on density-dependent regulation of pilosebin L4: for the gene encoding LanKC modification enzyme, LanKC-qF (5′-GGTTCCATCCTCTGATTGGTAGG-3′) and LanKC-qR (5′-CCCTCGTGACATTCCTCAAGC-3′); for the histidine kinase, HisK-qF (5′-CCAGTTGCGTTCCATCATCCA-3′) and HisK-qR (5′-GGAAGTCGATGTGGAGTCGG-3′); for the PaPak gene, a single copy house-keeping gene used as a normalizer, Pak-qF (5′-GCAACCCGACATCCTCATTA-3′) and Pak-qR (5′-AGTCGAAGAAGTCGCTCAGG-3′). The qPCR conditions used are: 1 cycle of 95° C. for 10 min, 50 cycles of 95° C. for 10 min, 57° C. for 30 sec, and 72° C. for 30 sec, and 1 cycle each of 95° C., 40° C., 55° C., and 99° C. for melting curve analyses. Three biological replicates were analyzed, with 3 technical replicates each.

### Mock Microbial Community Analysis

Fresh plate cultures of P. acnes HL030PA2, HL056PA1, HL086PA1, HL110PA3, and P. avidum HL083PV1 were harvested and sub-cultured in fresh BHI media to an OD595 of 1.0. The cultures were mixed in various ratios and spread evenly onto A-media plates. The plates were incubated anaerobically at 37° C. for 48 h. The mock communities were harvested, and genomic DNA was extracted using the Wizard® Genomic DNA Purification Kit (Promega) following the manufacturer's protocol for Gram Positive Bacteria. The community DNA was diluted to 20 ng/μL, and absolute quantity of each strain was analyzed using qPCR on the LightCycler® 480 System (Roche), with the LightCycler® 480 High Resolution Melting Dye (Roche) following the manufacturer's protocol. The following primer sets were used: for identification of HL056PA1, L1-qF (5′-GAAGAATCCCGCTCCATTTCC-3′) and L1-qR (5′-CCTTTCTTGTAGCCGAGCAG-3′); for identification of HL030PA2, HL086PA1, and P. avidum, HisK-qF (5′-CCAGTTGCGTTCCATCATCCA-3′) and HisK-qR (5′-GGAAGTCGATGTGGAGTCGG-3′); for identification of HL110PA3, cas3-qF (5′-GGCAAGACAAACGAGGTAGGAG-3′) and cas3-qR (5′-GATGGATTGTGGTTGGAGTCTCC-3′); for the Pak gene, a single copy house keeping gene found in all P. acnes, Pak-qF (5′-GCAACCCGACATCCTCATTA-3′) and Pak-qR (5′- AGTCGAAGAAGTCGCTCAGG-3′). The qPCR conditions used are: 1 cycle of 95° C. for 10 min, 50 cycles of 95° C. for 10 min, 57° C. for 30 sec, and 72° C. for 30 sec, and 1 cycle each of 95° C., 40° C., 55° C., and 99° C. for melting curve analyses. Three biological replicates were analyzed, with three technical replicates each.

### Results

### A Subset of P. acnes Strains Encode a Type III Lantibiotic

Non-core regions of all sequenced P. acnes strains were identified, which lead to the identified a genomic locus in RT8 strains of clade IB-1 and RT5 strains of clade IC (Tomida, 2013), which was named Locus 4. Using BAGELS (van Heel et al 2013), a class III lantibiotic gene cluster at the beginning of the Locus 4 in above mentioned clades IB-1 and IC strains were identified. The lantibiotic gene cluster contains 4 genes: the prepeptide, the modification gene LanKC, and a two-component system consisting of a histidine kinase and a response regulator (FIG. 1). A membrane transporter directly follows the lantibiotic gene cluster in locus 4, with homology to known drug efflux systems. Furthermore, an ABC transporter containing a domain subfamily involved in lantibiotic immunity is also encoded at the end of locus 4 (genes 1348 and 1349, FIG. 1).

### Locus 4 With and Without the Lantibiotic Operon is Found in Other Actinomycetales Species

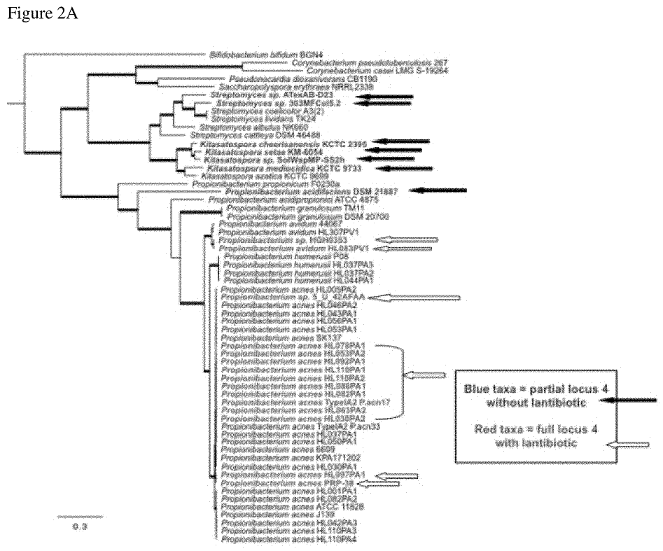

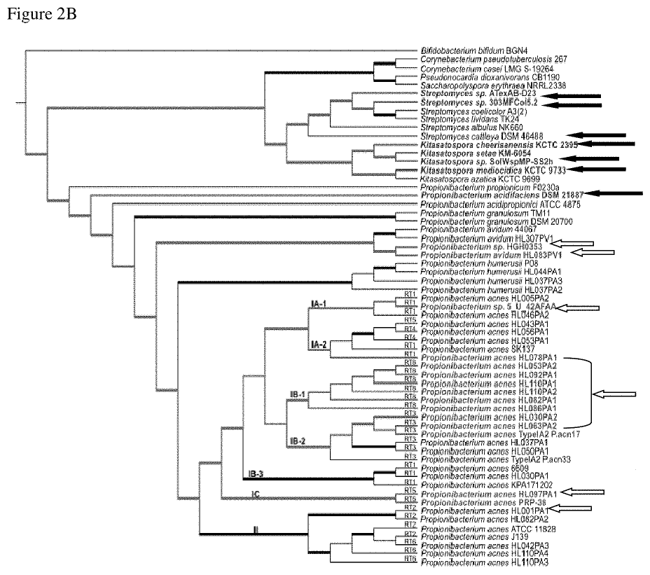

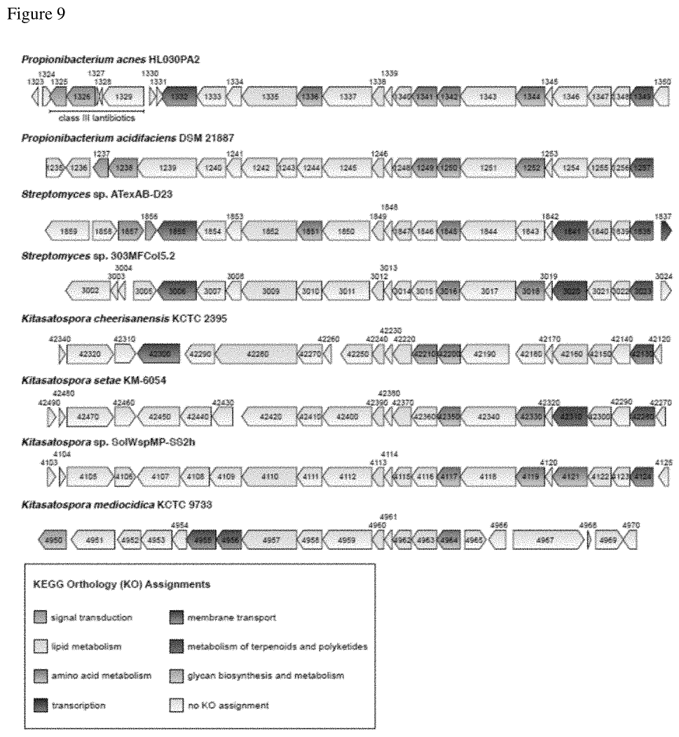

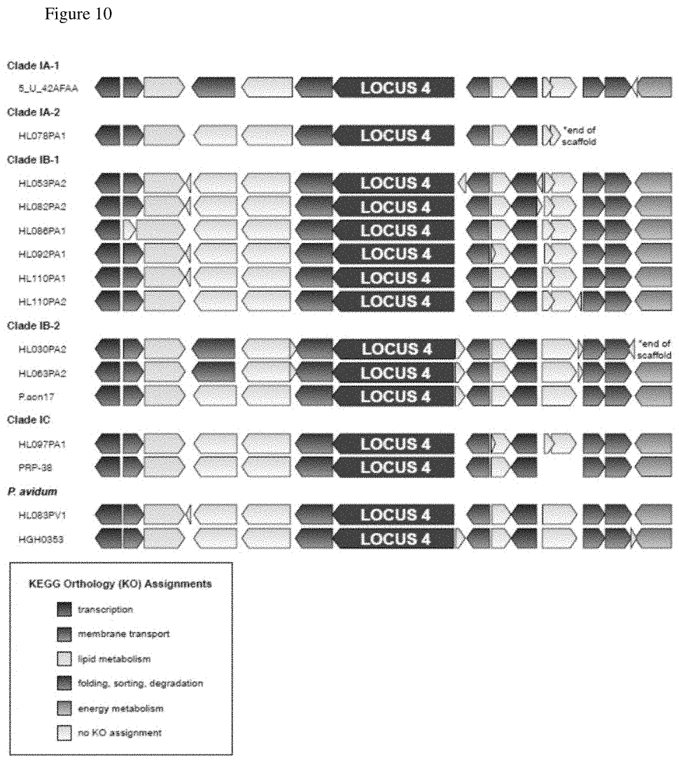

To determine if locus 4 containing the lantibiotic gene cluster is encoded in other bacteria, a BLAST search of the locus 4 DNA sequence was performed on all deposited bacterial sequences (including partial and draft sequences) on IMG (Markowitz et al 2012). The full locus 4 with the lantibiotic operon was detected in some P. avidum strains, including HGH0353 and HL083PV1 (FIG. 2A). Additionally, a partial locus 4 that lacks the lantibiotic operon was detected in other Actinomycetales species, specifically in the families of Streptomycetaceae and Propionibacteriaceae (FIG. 2A, FIG. 9). The full locus 4 regions in all the examined Propionibacterium strains are 99.8% identical, and are found in the same genomic location, indicating that this genetic island was likely acquired by an ancestor of P. acnes and P. avidum (FIG. 2B, FIG. 10). Only a small subset of Propionibacterium strains seems to have maintained this genetic locus, while the majority of strains have eliminated it from their genomes. In addition to clades IB-1 and IC, some strains from clade IB-2 (RT3) also harbor locus 4 (FIG. 2B). A BLAST search of the lantibiotic operon alone did not yield any results beyond the Propionibacterium species mentioned above. Since Propionibacterium species are the dominant residents in the pilosebaceous unit, and the lantibiotic genes are found in locus 4, this novel lantibiotic was named Pilosebin L4.

### Propionibacterium Species Harboring the Lantibiotic Can Inhibit a Broad-Range of Gram-Positive Species

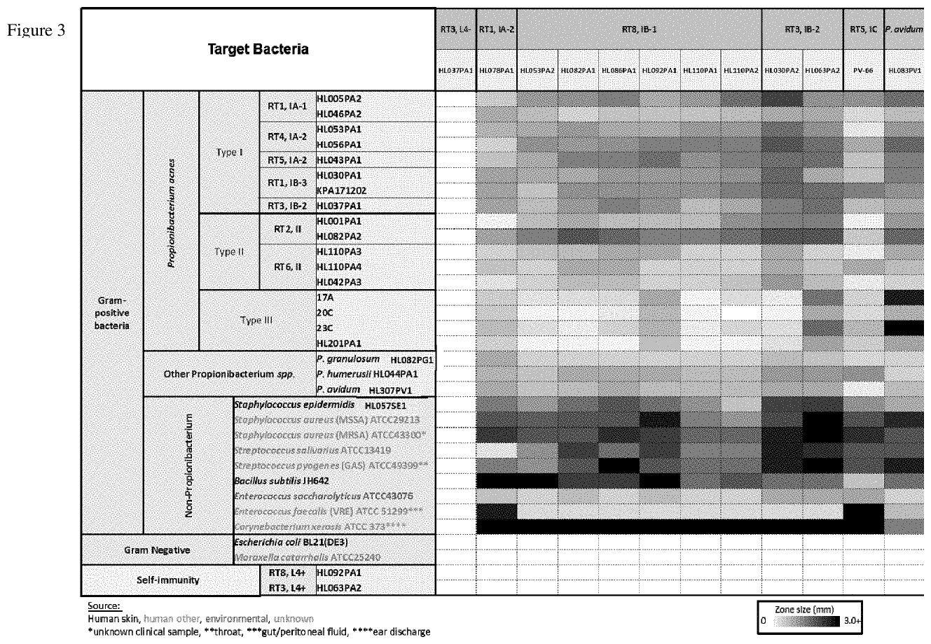

To determine if the lantibiotic has antimicrobial activity, an inhibition overlay assay was performed using 10 P. acnes strains (1 RT1 strain of clade IA-2, 6 RT8 strains of clade IB-1, 2 RT3 strains of clade IB-2, and 1 RT5 strain of clade IC) and 1 P. avidum strain that produce Pilosebin L4. The P. acnes strain HL037PA1 (RT3 from clade IB-2) was used as a negative control due to its phylogenetic closeness to the IB-1 and IB-2 strains that produce Pilosebin L4 (FIG. 2B). The ability of Pilosebin L4 to carry out strains to inhibit bacteria normally found on the skin and in the pilosebaceous unit. Pilosebin L4-negative P. acnes strains of all major lineages, a Pilosebin L4-negative P. avidum strain, P. granulosum, P. humerusii, S. epidermidis, and S. aureus were used as the target species and strains. All of the above bacteria were found in skin follicles (Fitz-Gibbon et al 2013). Pilosebin L4 producers were all able to inhibit the growth of the target species from the skin (FIG. 3). The RT3 strains from the IB-2 clade showed a slightly better inhibitory ability than RT8 strains from the IB-1 clade and P. avidum. Among the propionibacteria target strains, type I P. acnes strains were inhibited the most, while types II and III, and other Propionibacterium species (P. granulosum, P. humerusii, P. avidum) were inhibited less than P. acnes type I. The negative control strain P. acnes HL037PA1 was unable to inhibit any of the target strains. Pilosebin L4 producers were also unable to inhibit other Pilosebin L4 producers, indicating the presence of a functional lantibiotic immunity mechanism within locus 4 (FIG. 3).

The lantibiotic protects against a broad-range of Gram-positive species, including those of clinical importance, such as methicillin-resistant Staphylococcus aureus (MRSA), vancomycin-resistant enterococci (VRE, Enterococcus faecalis), group A streptococcus (GAS, Streptococcus pyogenes), and Clostridium difficile. Pilosebin L4 producers were able to inhibit the growth of all the species tested (FIGS. 3-5). Non-Propionibacterium species generally showed the greatest sensitivity to Pilosebin L4. Since lantibiotics are known to only be effective against Gram-positive species, two Gram-negative species, Escherichia coli and Moraxella catarrhalis, were tested and confirmed the functionality for Pilosebin L4. As expected, growth of the two Gram-negative species was not affected by Pilosebin L4.

### Pilosebin L4 is Regulated by Cell Density

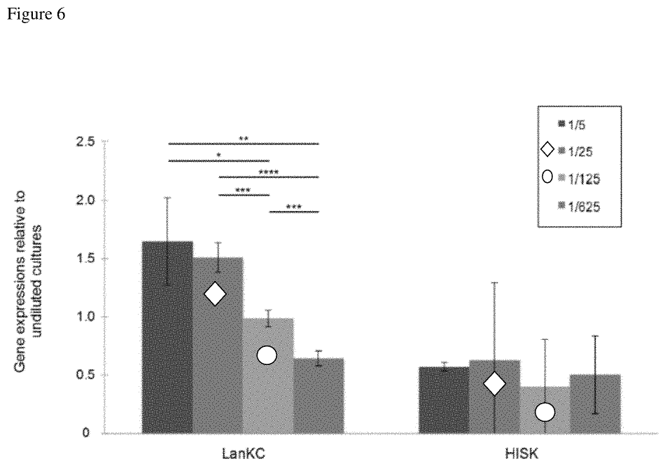

The presence of the two-component regulatory system in the lantibiotic operon (FIG. 1) led us to hypothesize that Pilosebin L4 is auto-regulated and affected by cell-density as in other lantibiotic systems (Hoover et al 2015, Lee et al 2011). Gene regulation by performing quantitative real-time PCR (qPCR) on the transcripts of the LanKC modification enzyme (locus tag HMPREF9602-01329) and the histidine kinase regulatory gene (locus tag HMPREF9602-01326) in P. acnes HL030PA2, at various cell densities. The LanKC modification gene expression was proportionate to cell density, where high cell density up-regulated transcription, and low cell density down-regulated transcription (FIG. 6).

### Pilosebin L4 Producers Can Dominate a Bacterial Community by Out-Competing Other Strains

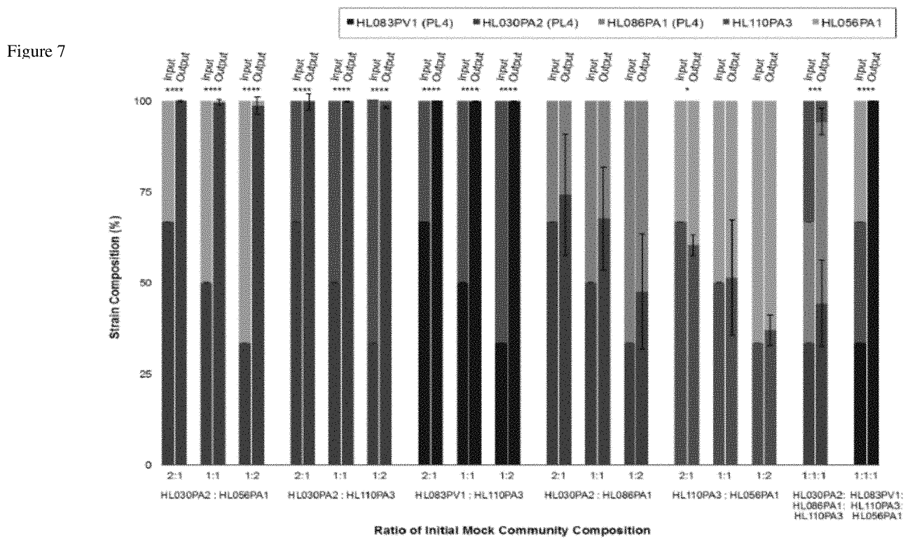

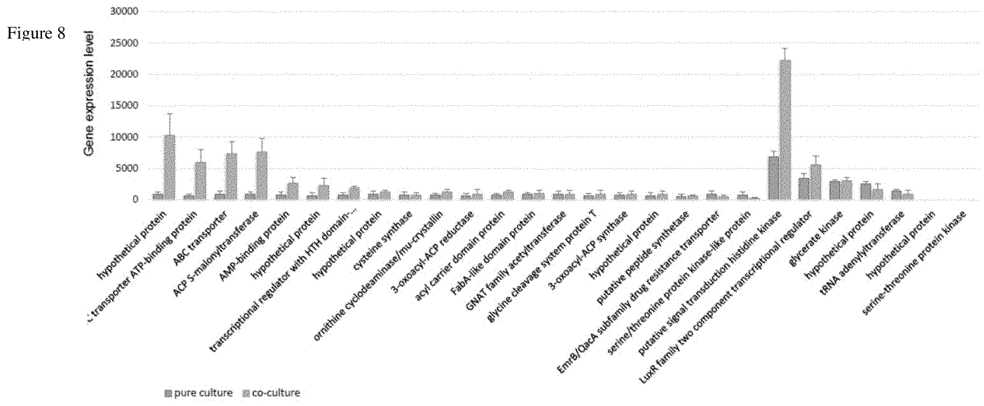

While the inhibition assays (FIG. 3) showed that the bacterial strains harboring Pilosebin L4 are able to inhibit Gram-positive species, the assay was performed in such way that the lantibiotic producers grow and release the lantibiotics prior to the placement of the target strains. Thus, mock bacterial communities were tested where both Pilosebin L4 producers and target strains were grown simultaneously, with no initial growth advantage of the producers. To assemble the mock communities, various combinations of two or three of the following strains in different ratios were grown: three Pilosebin L4 producing strains: P. acnes HL030PA2, P. acnes HL086PA1, and P. avidum HL083PV1, and two Pilosebin L4-negative strains: P. acnes HL056PA1 and P. acnes HL110PA3 (FIG. 7). The mock communities were monitored and the individual strain compositions of the mock communities were measured after 48 hours of growth using qPCR with strain-specific primers. In vitro mock communities showed that Pilosebin L4 producing P. acnes and P. avidum strains were able to significantly out-compete Pilosebin L4-negative strains, even if they were initially grown at lower proportions (FIG. 7). When either two Pilosebin L4 producing strains were grown together, or when two Pilosebin L4-negative strains were grown together, the composition of the mock communities remained similarly to the initial proportions. The output ratios between the strains showed no significant differences in five of the six sets, and only one set with marginal significant difference. This indicates that Pilosebin L4 provides a strong growth advantage in a mixed community to the producer strains over other Gram-positive strains that do not have Pilosebin L4 immunity. To further investigate at the molecular and transcriptional levels how Pilosebin L4 provides such growth advantage, a transcriptomic analysis comparing the gene expression of Pilosebin L4 positive P. acnes strains was performed in pure culture versus co-culture with other strains. As shown in FIG. 8, multiple genes encoded in locus 4 were significantly upregulated in co-cultures, suggesting that the presence of other strains or species signals the production of Pilosebin L4.

TABLE 1
Bacterial strains used in this study.

| | | | | | Culture | Incubation | |
|---|---|---|---|---|---|---|---|
| Species | Strain | Clade | Features | Isolation | media | condition | Source |
| Propionibacterium acnes | HL005PA2 | IA-1 | — | human skin | A | anaerobic, 37° C. | (Fitz-Gibbon et al 2013) |
| Propionibacterium acnes | HL046PA2 | IA-1 | — | human skin | A | anaerobic, 37° C. | (Fitz-Gibbon et al 2013) |
| Propionibacterium acnes | HL053PA1 | IA-2 | — | human skin | A | anaerobic, 37° C. | (Fitz Gibbon et al 2013) |
| Propionibacterium acnes | HL056PA1 | IA-2 | — | human skin | A | anaerobic, 37° C. | (Fitz-Gibbon et al 2013) |
| Propionibacterium acnes | HL043PA1 | IA-2 | — | human skin | A | anaerobic, 37° C. | (Fitz-Gibbon et al 2013) |
| Propionibacterium acnes | HL053PA2 | IB-1 | locus 4+ | human skin | A | anaerobic, 37° C. | (Fitz-Gibbon et al 2013) |
| Propionibacterium acnes | HL082PA1 | IB-1 | locus 4+ | human skin | A | anaerobic, 37° C. | (Fitz-Gibbon et al 2013) |
| Propionibacterium acnes | HL086PA1 | IB-1 | locus 4+ | human skin | A | anaerobic, 37° C. | (Fitz-Gibbon et al 2013) |
| Propionibacterium acnes | HL092PA1 | IB-1 | locus 4+ | human skin | A | anaerobic, 37° C. | (Fitz-Gibbon et al 2013) |
| Propionibacterium acnes | HL110PA1 | IB-1 | locus 4+ | human skin | A | anaerobic, 37° C. | (Fitz Gibbon et al 2013) |
| Propionibacterium acnes | HL110PA2 | IB-1 | locus 4+ | human skin | A | anaerobic, 37° C. | (Fitz-Gibbon et al 2013) |
| Propionibacterium acnes | HL030PA2 | IB-2 | locus 4+ | human skin | A | anaerobic, 37° C. | (Fitz-Gibbon et al 2013) |
| Propionibacterium acnes | HL063PA2 | IB-2 | locus 4+ | human skin | A | anaerobic, 37° C. | (Fitz-Gibbon et al 2013) |
| Propionibacterium acnes | HL037PA1 | IB-2 | — | human skin | A | anaerobic, 37° C. | (Fitz-Gibbon et al 2013) |
| Propionibacterium acnes | HL030PA1 | IB-3 | — | human skin | A | anaerobic, 37° C. | (Fitz-Gibbon et al 2013) |
| Propionibacterium acnes | KPA171202 | IB-3 | — | human skin | A | anaerobic, 37° C. | (Fitz-Gibbon et al 2013) |
| Propionibacterium acnes | HL001PA1 | II | — | human skin | A | anaerobic, 37° C. | (Fitz-Gibbon et al 2013) |
| Propionibacterium acnes | HL082PA2 | II | — | human skie | A | anaerobic, 37° C. | (Fitz-Gibbon et al 2013) |
| Propionibacterium acnes | HL042PA3 | II | — | human skin | A | anaerobic, 37° C. | (Fitz-Gibbon et al 2013) |
| Propionibacterium acnes | HL110PA3 | II | — | human skin | A | anaerobic, 37° C. | (Fitz Gibbon et al 2013) |
| Propionibacterium acnes | HL110PA4 | II | — | human skin | A | anaerobic, 37° C. | (Fitz-Gibbon et al 2013) |
| Propionibacterium granulosum | HL082PG1 | n/a | — | human skin | A | anaerobic, 37° C. | (Fitz-Gibbon et al 2013) |
| Propionibacterium humerusii | HL044PA1 | n/a | — | human skin | A | anaerobic, 37° C. | |
| Propionibacterium avidum | HL307PV1 | n/a | — | human skin | A | anaerobic, 37° C. | |
| Propionibacterium avidum | HL083PV1 | n/a | locus 4+ | human skin | A | anaerobic, 37° C. | |
| Staphylococcus epidermidis | HL057SE1 | n/a | — | human skin | A | aerobic, 37° C. | |
| Staphylococcus aureus | F-182 | n/a | MRSA | human, other | BHI | aerobic, 37° C. | ATCC 43300 |
| Streptococcus salivarius | C699 | n/a | — | unknown | BHI | aerobic, 37° C. | ATCC 13419 |
| Streptococcus pyogenes | QC A62 | n/a | Group A | human, other | BHI | aerobic, 37° C. | ATCC 49399 |
| | | | Strep | | | | |
| Bacillus subtilis | JH642 | n/a | — | | BHI | aerobic, 37° C. | |
| Commebacterium xerosis | | n/a | — | human, other | BHI | aerobic, 37° C. | ATCC 373 |
| Enterococcus saccharolyticus | NCDO 2594 | n/a | — | environment | BHI | aerobic, 37° C. | ATCC 43076 |
| Enterococcus faecalis | NJ-3 | n/a | VRE | human, other | BHI | aerobic, 37° C. | ATCC 51299 |
| Escherichia coli | BL21(DE3) | n/a | — | lab strain | BHI | aerobic, 37° C. | |
| Moraxella catarrhalis | N9 | n/a | — | unknown | BHI | aerobic, 37° C. | ATCC 25240 |

Fitz-Gibbon S, Tomida S, Chiu BH, Nguyen L, Du C, Liu M et al (2013).
Propionibacterium acnes strain populations in the human skin microbiome associated with acne. J Invest Dermatol 133: 2152-2160.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments are described herein. Such equivalents are intended to be encompassed by the following claims.

What is claimed is:

1. A method of treating or preventing an infection caused by Gram-positive bacteria in a subject in need thereof, comprising administering a composition comprising a Propionibacterium that comprises the nucleic acid sequence of SEQ ID NO.: 6 or SEQ ID NO.: 15 to the subject.

2. The method of claim 1, wherein the Propionibacterium is P. acnes.

3. The method of claim 2, wherein the Propionibacterium acnes bacterium is a P. acnes strain selected from HL037PA1, HL078PA1, HL082PA1, HL086PA1, HL092PA1, HL110PA1, HL030PA2, HL030PA1, HL063PA2, PV-66, Type IA2 P.acn17, HL097PA1, PRP-38, 5_U_42AFAA, and HL082PA2.

4. The method of claim 1, wherein the Propionibacterium is P. avidum.

5. The method of claim 4, wherein the Propionibacterium avidum is P. avidum strain HL083PV1 or HGH0353.

6. The method of claim 1, wherein the Propionibacterium encodes for the amino acid sequence of any one of SEQ ID NO.: 1-5 or 7-14.

7. The method of claim 1, wherein the composition is heat-treated or tyndallized.

8. The method of claim 1, wherein the composition is formulated for topical delivery.
